SEQUENCE LISTING (13 sequences)

SEQ ID NO: 1 - DNA, 1278 bp, Caenorhabditis elegans
atgcctcgaatcaccgccagtaaaatagttcttctaattgcattatcattttgtattactgttatttatcactttccaatagcaacgagaagcagtaaggagtacgatgaatatggaaatgaatatgaaaacgttgcatcgatagagtcggatataaaaaatgtacgtcgattacttgacgaggtaccggatccctcacaaaaccgtctacaattcctgaaacttgatgagcatgcttttgcattctcggcctacacagacgatcgaaatggaaatatggggtacaaatatgtccgagtcctgatgtttatcacgtcacaagacaacttttcctgtgaaataaacgggagaaagtccacagatgtatcactttacgagttctcggaaaatcacaaaatgaagtggcaaatgtttattttgaattgtaaactacccgatggtatagatttcaataatgttagctctgtaaaggtcataagaagcacaaccaagcagtttgttgatgtgccgattcggtatagaattcaagatgagaaaataattacgccagacgaatatgactataaaatgtcaatttgtgttccagcattgtttggaaatggatatgatgcaaagcgaattgttgagtttattgagctgaatactttgcaaggaatcgagaaaatatacatttacactaatcaaaaagagcttgatggatccatgaagaaaacgttgaaatactattcggataatcacaaaataacattaattgattacacattaccattcagagaggatggtgtttggtatcacgggcaattggcaactgttactgattgtttactgagaaacactggaatcacaaaatacacatttttcaatgattttgatgagttcttcgtccccgttatcaaaagtcggactctctttgaaacaatcagtgggctttttgaagatcccactattggatcgcaacgaacagctttgaagtatataaatgcaaaaatcaagagcgctccgtattcactgaaaaatattgtttccgaaaaacgaattgaaacaagattcacgaaatgtgtagttcgaccggaaatggtttttgaacagggtattcatcatacgagtagagtgattcaagacaactataaaacggtttcccatggcggatcccttctacgggtttatcattacaaggataaaaagtattgttgcgaagacgagagcctcttgaaaaaacggcatggagatcaacttcgggaaaaattcgattcagttgttggtcttttagacttgtag

SEQ ID NO: 2 - protein, 425 aa, Caenorhabditis elegans
MPRITASKIVLLIALSFCITVIYHFPIATRSSKEYDEYGNEYENVASIESDIKNVRRLLDEVPDPSQNRLQFLKLDEHAFAFSAYTDDRNGNMGYKYVRVLMFITSQDNFSCEINGRKSTDVSLYEFSENHKMKWQMFILNCKLPDGIDFNNVSSVKVIRSTTKQFVDVPIRYRIQDEKIITPDEYDYKMSICVPALFGNGYDAKRIVEFIELNTLQGIEKIYIYTNQKELDGSMKKTLKYYSDNHKITLIDYTLPFREDGVWYHGQLATVTDCLLRNTGITKYTFFNDFDEFFVPVIKSRTLFETISGLFEDPTIGSQRTALKYINAKIKSAPYSLKNIVSEKRIETRFTKCVVRPEMVFEQGIHHTSRVIQDNYKTVSHGGSLLRVYHYKDKKYCCEDESLLKKRHGDQLREKFDSVVGLLDL

SEQ ID NO: 3 - DNA, 1311 bp, Caenorhabditis briggsae
atgccacgaataacggcaagcaaaatagtgttattatctgtattatccttactaacagttttctatctgaatacattttcgtctattaaaattgaaaacgatctcgacgggactgattacgacttggattacatagaatctgatatcaaaaagacgcgtcgattactcaatgaaatccctgatccatctcaaaaccgagttcaattttttaaactcgatgataatggatatgcattctcagcatatacagataataggaaaggaaatatgggtcacaaatatgtcagaatattagtgttcctaactaaatttgatgatttttcttgcgaaattaactcgaagaaatcctatgttgttacactctacgagctatcagaaaatcacaatatgaagtggaaaatgtatattttgaattgtttacttcccgatggaatcactttcaacgatgtgaattctgtaaaaatatctagaagttcttcaaaactttcagtccaaatcccgatcagatatagaattcaagatgagaaaatgatgactccagatgaatacgattataagttgtcgatttgtgttcctgcactttttggaaacgtttattatccaaggaggattattgaatttgtggaactaaacagcttgcaagacatcgacaaaatctacatctactacaatcctttagaaatgacagatgaggccacagaaaggactttgaagttttattccaataatgggaaaatcaatttaatagaattcattctcccattttctactcgagatgtttggtattatgggcaattggccaccgttacagattgtcttctccgtaacactggaataactcaatacacatttttcaatgatttggatgaatttttcgtgccagtactggacaaccaaactctctctgaaactgtgtcaggattatttgaaaatcgaaaaattgcctctcagagaacggccttgaaatttattagtacaaaaatcaatcgatctcctgtaactctcaataatattgtgtcttctaaaaattttgaaacgagattcacaaaatgcgtcgtacggccggaaatggtttttgagcagggcattcaccatacgagtagagtaatacaagacgactacgaaaccccatcccatgatggatcacttttgcgtgtgtatcactacagagaaccaagatattgctgcgaaaacgagaatcttctaaaacaaagatacgataagaagcttcaagaagtttttgatgctgtagttcttatattgcatgtcacatttgatgtatggatatatcacctgaaaaacaccctctaa

SEQ ID NO: 4 - protein, 436 aa, Caenorhabditis briggsae
MPRITASKIVLLSVLSLLTVFYLNTFSSIKIENDLDGTDYDLDYIESDIKKTRRLLNEIPDPSQNRVQFFKLDDNGYAFSAYTDNRKGNMGHKYVRILVFLTKFDDFSCEINSKKSYVVTLYELSENHNMKWKMYILNCLLPDGITFNDVNSVKISRSSSKLSVQIPIRYRIQDEKMMTPDEYDYKLSICVPALFGNVYYPRRIIEFVELNSLQDIDKIYIYYNPLEMTDEATERTLKFYSNNGKINLIEFILPFSTRDVWYYGQLATVTDCLLRNTGITQYTFFNDLDEFFVPVLDNQTLSETVSGLFENRKIASQRTALKFISTKINRSPVTLNNIVSSKNFETRFTKCVVRPEMVFEQGIHHTSRVIQDDYETPSHDGSLLRVYHYREPRYCCENENLLKQRYDKKLQEVFDAVVLILHVTFDVWIYHLKNTL

SEQ ID NO: 5 - DNA, 1499 bp, Nematostella vectensis
atgcgatgctatatttacaaattgaggttgtccgtttgtctgtttgtagtgctcttcacagcactgcttttcatcacctatttaaaccactcagagcttgaatcagcagagaaaagtagcggaaaaaggaagacgcgacatcgtaaacgaacacgttcacgcaaacaacacgagagccattttcagaaagctcgactacaagaaagagaactagtattaagatctacagcgccaccaacattacgaagagaagtacaagcgcatcgattagggcagatccgtggcaagaacacggaccaggggataactggaaagttcacagagatcgctaaagacacgcatatttattcagcgttttacgacgatgccaagtcaaatccattcattcgtcttatcatcctctcgggaaaacactaccagcctggattatcttgccaattttgcgaacctttgtccgccagttgtagttttgcggactctaaagctgaatactacacgaccaacgagaaccatgggagagtatttggcgggttcattgcgagttgcctcgtgcctgatggattcaatgcagtgccattgtttgttgacataacggccgatgttaagggggagaaaagcaaggcacgggtacctgtggtgtctaatgcacatctctactaccctattaaatacgcaatctgcgtcccacccctccgatcagagaaactaacagcgaaaagactcatagagtttgtcgagctaaccaaacttttaggcgctaaccattttactttttatgacttcaaaacggacccggaagtcaataacgttttaagatattaccaggagacacaagtagcaaatgttctgccatggaatctaccttcaaatttggtatccaggccgaacgatatttggtactttggtcaggttttggctattctagattgcttgtatcgctacaagaacagggcaaaatttgtagccttcaatgacgtagatgagtttatcgttccgctaaggaacagctcgatagtggaaatactaaacgcgtttcaccggccataccactgtggacattgctttcagagcgtggtgttcagctcaaacgcgagatttcccaggcaaaaaagcgagttagtttctcagcggttcttccacaggacccaggaaaccatccctctcctctcgaaatgcattgtggatcctttgagagtgttcgagatggggattcaccacataagcaaggctacaggtctgcggtattccgtcaactcagtacacgagagtgacgcggttatcttccattacaggacttgcactacgtcatttggtatacgtcatcagtgcatgaacctagtgcatgatgggaccatggccaaatatggaaaacgacttcagaaaatgtttagaaaggttgtaaatgatttaaaacttttggcaccaacgtagctatttcgtaacacttcacactttcattgttataacagaatacagaataaattaatgattgttgtgcc

SEQ ID NO: 6 - protein, 476 aa, Nematostella vectensis
MRCYIYKLRLSVCLFVVLFTALLFITYLNHSELESAEKSSGKRKTRHRKRTRSRKQHESHFQKARLQERELVLRSTAPPTLRREVQAHRLGQIRGKNTDQGITGKFTEIAKDTHIYSAFYDDAKSNPFIRLIILSGKHYQPGLSCQFCEPLSASCSFADSKAEYYTTNENHGRVFGGFIASCLVPDGFNAVPLFVDITADVKGEKSKARVPVVSNAHLYYPIKYAICVPPLRSEKLTAKRLIEFVELTKLLGANHFTFYDFKTDPEVNNVLRYYQETQVANVLPWNLPSNLVSRPNDIWYFGQVLAILDCLYRYKNRAKFVAFNDVDEFIVPLRNSSIVEILNAFHRPYHCGHCFQSVVFSSNARFPRQKSELVSQRFFHRTQETIPLLSKCIVDPLRVFEMGIHHISKATGLRYSVNSVHESDAVIFHYRTCTTSFGIRHQCMNLVHDGTMAKYGKRLQKMFRKVVNDLKLLAPT

SEQ ID NO: 7 - DNA, 1260 bp, Taeniopygia guttata
atgactgtaactttaatgcttgtggtttcttatctgagattacagagactttctcatcagccaaaagtaattcaagaaagtagaagatgtagagggaaaattgcccttagcacaataacagcattggaaggtaacaaaactgatattatatccccatactttgatgacagagaaaacaaaatcactcgtctgattgggattgttcaccataaagatgtaaaacaactgttctgctggttctgctgtcaagccaatggaaagatatatgtatcaaaagcagaaatagatgttcactcggatagatttggattcccttatggtgcagcagatataatttgtttggaacctgaaaactgtgatccaacacatgtatcaattcatcagtctccatatggaaatattgaccagctgccgaggtttgaaattaaaaatcgcaggcctgagaccttttctgttgacttcaccgtgtgcatttctgccatgtttggaaactacaacaatgtcttgcagtttgtacagagtatggaaatgtataagattcttggagtacagaaagtggtgatctataagaacaactgcagccatctgatggagaaagtcttgaaattttatatagaagaaggaactgttgaggtaattccctggccaatagactcacacctcagggtttcttctaaatggcgcttcatggaagacgggacacacattggctactatggacaaatcacagctctaaatgactgtatataccgcaacatggaaaggaccaagtttgtggtccttaatgacgctgatgaaataattcttccccttaaacacccagactggaaaacaatgatgaacagtcttcaggagcaaaacccagggactagtgttttcctttttgagaaccatatcttcccagaaactgtattttctcccatgttcaacatttcatcttggaatactgtgccaggtgttaacatattgcagcatgtgtacagagagcctgacaggaaacatgtaatcaatcccaggaaaatgatagttgatccacgaaaggtgattcagacttcagtccattctgtcctacgtgcttatgggaagagcgtgaatgttcccatggaagttgccctcatttatcactgtcggaaggcccttcaaggaaaccttcccagagaatctctcatcagggatacaacactgtggagatataactcatcattaatcatgaatgttaacaaggttctatctcaaaccatgctgcaaactcaaaattga

SEQ ID NO: 8 - protein, 419 aa, Taeniopygia guttata
MTVTLMLVVSYLRLQRLSHQPKVIQESRRCRGKIALSTITALEGNKTDIISPYFDDRENKITRLIGIVHHKDVKQLFCWFCCQANGKIYVSKAEIDVHSDRFGFPYGAADIICLEPENCDPTHVSIHQSPYGNIDQLPRFEIKNRRPETFSVDFTVCISAMFGNYNNVLQFVQSMEMYKILGVQKVVIYKNNCSHLMEKVLKFYIEEGTVEVIPWPIDSHLRVSSKWRFMEDGTHIGYYGQITALNDCIYRNMERTKFVVLNDADEIILPLKHPDWKTMMNSLQEQNPGTSVFLFENHIFPETVFSPMFNISSWNTVPGVNILQHVYREPDRKHVINPRKMIVDPRKVIQTSVHSVLRAYGKSVNVPMEVALIYHCRKALQGNLPRESLIRDTTLWRYNSSLIMNVNKVLSQTMLQTQN

SEQ ID NO: 9 - DNA, 1647 bp, Cryptosporidium parvum
atgcaaagtaaagtcatttttaggatcttggtattgatcatttcggtgattggatccttatactcaataattcaattaatgctaaaggagctatcaagtaacaaaaatattcaagaggttagtcattcaaggaggctaataagtgaaccttacagtgaaagtattaatgaacaaaatgatcaagattggaaagaactaaagctaataattccaaatcattctcaaattaaccagcaggaaaaaaatggtaatttgattgagtttaaagtttatatatactcagcatattatgattggagaatagataggatacgaataaattcacttatcccatcgaatttttatgatcgaatagaaatggaatgtgcaataatcttggacaaaaatatttacacaggaactattaaaaaagtgattcataaggagcaccataataaagaatatgtatcatcgactttactctgcgaaattgcaaaaaatgaaattaaatttgaggatatttcaaggaaagttttgataacaattttggaaaatggaaacagcacaaataaatcagaaatatggataactctaaaaaaaattccaaaaaatagctctaataatcatgagctgactgtttgtgtgagaccttggtggggagagccaataaagaatggaaacttgggaaataaacaaaaatttaacaattcagggttaatgcttgaatttattaattcatatttattcttaggagcaaataaattttatttatatcaaaattacttggacattgacgaagatgtaagaaatataataaattattattctaatatcaaaaatgttttggaaattattccatactcattaccaataattccatttaaacaagtttgggatttcgcacaaacaacaatgatacaggactgcctactaagaaatattggaaaaacaaaatacttgttattcgtagataccgatgaatttgtatttccaaacttgaaaaattataacttaatggattttttaaatttattagaagccaacaatccttattataaaaacaaagtcggggcaatgtggattccaatgtattttcattttttagagtgggaatctgataaaaataatttgaagaaatattcaacaattgagaaaaaaattaagaaaaagatggcaaatattgagtttgttctatatcgtaaaacatgtagaatgttaagttctggaacaaaaaaaagtgacaagacgagaagaaaagttattattagacctgaaagagttttgtatatgggtatacatgaaacagaagagatgctaagcaaaaaatttcatttcattagagctcctgtaattaatgtgggtggaggaaacgaactaagtatatatttacatcattatagaaaagcaaaaggtattgtaaacaatgatcccaaacaaagagaacttgtgaatatgtatttagaaaatgtttgttcagataagctgttagattcagggggagattccattcaagatggagtaattgtcgacaatactgtttgggagatatttggaacacacttataccagataatttttgagcatattaaagaaatccaagatatgtacacaaataaggaaataattaatggaaataaaaatttaagtgttgaagaattacataattaa

SEQ ID NO: 10 - protein, 548 aa, Cryptosporidium parvum
MQSKVIFRILVLIISVIGSLYSIIQLMLKELSSNKNIQEVSHSRRLISEPYSESINEQNDQDWKELKLIIPNHSQINQQEKNGNLIEFKVYIYSAYYDWRIDRIRINSLIPSNFYDRIEMECAIILDKNIYTGTIKKVIHKEHHNKEYVSSTLLCEIAKNEIKFEDISRKVLITILENGNSTNKSEIWITLKKIPKNSSNNHELTVCVRPWWGEPIKNGNLGNKQKFNNSGLMLEFINSYLFLGANKFYLYQNYLDIDEDVRNIINYYSNIKNVLEIIPYSLPIIPFKQVWDFAQTTMIQDCLLRNIGKTKYLLFVDTDEFVFPNLKNYNLMDFLNLLEANNPYYKNKVGAMWIPMYFHFLEWESDKNNLKKYSTIEKKIKKKMANIEFVLYRKTCRMLSSGTKKSDKTRRKVIIRPERVLYMGIHETEEMLSKKFHFIRAPVINVGGGNELSIYLHHYRKAKGIVNNDPKQRELVNMYLENVCSDKLLDSGGDSIQDGVIVDNTVWEIFGTHLYQIIFEHIKEIQDMYTNKEIINGNKNLSVEELHN

SEQ ID NO: 11 - DNA, 25 bp, Artificial Sequence
  PCR primer
tttgtcgacacttctgaatgcctcg

SEQ ID NO: 12 - DNA, 30 bp, Artificial Sequence
  PCR primer
ttttctagactacaagtctaaaagaccaac

SEQ ID NO: 13 - DNA, 24 bp, Artificial Sequence
  PCR primer
tttgtcgaccctcgaatcaccgcc

(19) United States
(12) Patent Application Publication
(10) Pub. No.: US 2015/0203828 A1
(43) Pub. Date: Jul. 23, 2015

(54) N-GLYCAN CORE BETA-GALACTOSYLTRANSFERASE AND USES THEREOF

(71) Applicants: ETH ZURICH, Zurich (CH); UNIVERSITAT FUR BODENKULTUR WIEN, Vienna (AT); UNIVERSITAT ZURICH, Zurich (CH)

(72) Inventors: Markus KÜNZLER, Neuenhof (CH); Markus AEBI, Wettingen (CH); Iain WILSON, Vienna (AT); Alexander Walter TITZ, Kreuzlingen (CH); Micahel HENGARTNER, Zurich (CH); Alex BUTSCHI, Hinwil (CH)

(73) Assignees: ETH ZURICH, Zurich (CH); UNIVERSITAT FUR BODENKULTUR WIEN, Vienna (AT); UNIVERSITAT ZURICH, Zurich (CH)

(21) Appl. No.: 14/186,083

(22) Filed: Feb. 21, 2014

Related U.S. Application Data
(62) Division of application No. 13/322,505, filed on Nov. 25, 2011, now abandoned.

(30) Foreign Application Priority Data
May 28, 2009 (EP) 09007139.0

(51) Int. Cl. C12N 9/10

(52) U.S. Cl. CPC C12N 9/1051

(57) ABSTRACT

The present invention relates to new galactosyltransferases, nucleic acids encoding them, as well as recombinant vectors, host cells, antibodies, uses and methods relating thereto.

The present invention relates to new galactosyltransferases, nucleic acids encoding them, as well as recombinant vectors, host cells, antibodies, uses and methods relating thereto.

The “roundworms” or “nematodes” are the most diverse phylum of pseudocoelomates and one of the most diverse of all animals. Nematode species are difficult to distinguish; over 80,000 have been described, of which over 15,000 are parasitic. It has been estimated that the total number of roundworm species might be more than 500,000. Nematodes are ubiquitous in freshwater, marine and terrestrial environments. The many parasitic forms include pathogens in most plants, animals and also in humans.

Caenorhabditis elegans is a model nematode and is unsegmented, vermiform, bilaterally symmetrical, with a cuticle integument, four main epidermal cords and a fluid-filled pseudocoelomate cavity. In the wild, it feeds on bacteria that develop on decaying vegetable matter. Hannemann et al. (Glycobiology, 16, 874, 2006) isolated and structurally characterized D-galactopyranosyl-β-1,4-L-fucopyranosyl-α-1,6-D-GlcNAc (Gal-Fuc) epitopes at the core of N-glycans from Caenorhabditis elegans. The N-glycosylation pattern of Caenorhabditis elegans was recently reviewed in Paschinger et al. (Carbohydrate Res., 343, 2041, 2008).

It is the object of the present invention to provide new means for the recombinant production of Gal-Fuc-containing (poly/oligo)saccharides and Gal-Fuc-containing glycoconjugates. An additional object is to provide new uses for Gal-Fuc-containing poly/oligosaccharides and Gal-Fuc-containing glycoconjugates.

In a first aspect, the object is solved by an isolated and purified nucleic acid selected from the group consisting of:
(i) a nucleic acid comprising at least a nucleic acid sequence selected from the group consisting of nucleic acid sequences listed in SEQ ID NOs: 1, 3, 5, 7 and 9, preferably SEQ ID NO 1;
(ii) a nucleic acid having a sequence of at least 60, 65, 70 or 75% identity, preferably at least 80, 85 or 90% identity, more preferred at least 95% identity, most preferred at least 98% identity with a nucleic acid sequence selected from the group consisting of nucleic acid sequences listed in SEQ ID NOs 1, 3, 5 and 7, preferably SEQ ID NO: 1;
(iii) a nucleic acid that hybridizes to a nucleic acid of (i) or (ii);
(iv) a nucleic acid, wherein said nucleic acid is derivable by substitution, addition and/or deletion of one of the nucleic acids of (i), (ii) or (iii);
(v) a fragment of any of the nucleic acids of (i) to (iv), that hybridizes to a nucleic acid of (i).

In a preferred aspect the isolated and purified nucleic acid selected from the group consisting of:
(i) a nucleic acid comprising at least a nucleic acid sequence selected from the group consisting of nucleic acid sequences listed in SEQ ID NOs: 1, 3, 7 and 9 as well as the first 1428 nucleic acids of SEQ ID NO: 5, preferably SEQ ID NO 1;
(ii) a nucleic acid having a sequence of at least 60, 65, 70 or 75% identity, preferably at least 80, 85 or 90% identity, more preferred at least 95% identity, most preferred at least 98% identity with a nucleic acid sequence selected from the group consisting of nucleic acid sequences listed in SEQ ID NOs 1, 3 and 7 as well as the first 1428 nucleic acids of SEQ ID NO: 5, preferably SEQ ID NO: 1;
(iii) a nucleic acid that hybridizes to a nucleic acid of (i) or (ii);
(iv) a nucleic acid, wherein said nucleic acid is derivable by substitution, addition and/or deletion of one of the nucleic acids of (i), (ii) or (iii);
(v) a fragment of any of the nucleic acids of (i) to (iv), that hybridizes to a nucleic acid of (i).

Preferably, the above nucleic acids encode a polypeptide of the invention, preferably one having an enzymatic galactosyltransferase activity, more preferably one having a β-1,4-galactosyltransferase activity, preferably one with L-fucoside-, more preferably one with α-L-fucoside-, more preferably one with Fuc-α-1,6-GlcNAc- and most preferably one with GnGnF⁶- (nomenclature according to Schachter, Biochem. Cell. Biol. 64(3), 163-181, 1986) containing poly/oligosaccharides or glycoconjugates as acceptor substrates.

Galactosyltransferase activity, as used herein, is meant to describe an enzymatic transfer of a galactose residue from an activated donor form (i.e. nucleotide-activated galactose, preferably UDP-Gal) to an acceptor. β-1,4-Galactosyltransferase activity, as used herein, is meant to describe the specificity of the galactosyltransferase activity, i.e the transfer of galactose in a beta 1,4-configuration onto an acceptor molecule. β-1,4-Galactosyltransferase activity on L-fucosides as acceptor substrate, as used herein, is meant to describe the specificity of the galactosyltransferase activity in a beta-linked 1,4-transfer onto L-fucosides as the acceptor substrate. L-fucosides, as meant herein, are meant to describe poly/oligosaccharides or glycoconjugates as acceptor substrates containing terminal L-fucose in alpha, most preferably in alpha-1,6 configuration, e.g. as part of MMF6 or GnGnF⁶ (Schachter, Biochem. Cell. Biol. 64(3), 163-181, 1986).

In a most preferred embodiment, the encoded polypeptide comprises a polypeptide sequence selected from the group consisting of polypeptide sequences listed in SEQ ID NOs 2, 4, 6, 8 and 10, preferably SEQ ID NO: 2, or a functional fragment or functional derivative of any of these.

SEQ ID NO: 1 is the nucleic acid sequence coding for SEQ ID NO 2: (also listed in NCBI as Ref Seq NM_{—}072144.4 and in Wormbase as M03F8.4; coding for galactosyltransferase [referred to as GalT in the Examples section] from Caenorhabditis elegans)

ATGCCTCGAATCACCGCCAGTAAAATAGTTCTTCTAATTGCATTATCATT
TTGTATTACTGTTATTTATCACTTTCCAATAGCAACGAGAAGCAGTAAGG
AGTACGATGAATATGGAAATGAATATGAAAACGTTGCATCGATAGAGTCG
GATATAAAAAATGTACGTCGATTACTTGACGAGGTACCGGATCCCTCACA
AAACCGTCTACAATTCCTGAAACTTGATGAGCATGCTTTTGCATTCTCGG
CCTACACAGACGATCGAAATGGAAATATGGGGTACAAATATGTCCGAGTC
CTGATGTTTATCACGTCACAAGACAACTTTTCCTGTGAAATAAACGGGAG
AAAGTCCACAGATGTATCACTTTACGAGTTCTCGGAAAATCACAAAATGA
AGTGGCAAATGTTTATTTTGAATTGTAAACTACCCGATGGTATAGATTTC
AATAATGTTAGCTCTGTAAAGGTCATAAGAAGCACAACCAAGCAGTTTGT
TGATGTGCCGATTCGGTATAGAATTCAAGATGAGAAAATAATTACGCCAG
ACGAATATGACTATAAAATGTCAATTTGTGTTCCAGCATTGTTTGGAAAT
GGATATGATGCAAAGCGAATTGTTGAGTTTATTGAGCTGAATACTTTGCA
AGGAATCGAGAAAATATACATTTACACTAATCAAAAAGAGCTTGATGGAT
CCATGAAGAAAACGTTGAAATACTATTCGGATAATCACAAAATAACATTA
ATTGATTACACATTACCATTCAGAGAGGATGGTGTTTGGTATCACGGGCA
ATTGGCAACTGTTACTGATTGTTTACTGAGAAACACTGGAATCACAAAAT
ACACATTTTTCAATGATTTTGATGAGTTCTTCGTCCCCGTTATCAAAAGT
CGGACTCTCTTTGAAACAATCAGTGGGCTTTTTGAAGATCCCACTATTGG
ATCGCAACGAACAGCTTTGAAGTATATAAATGCAAAAATCAAGAGCGCTC
CGTATTCACTGAAAAATATTGTTTCCGAAAAACGAATTGAAACAAGATTC
ACGAAATGTGTAGTTCGACCGGAAATGGTTTTTGAACAGGGTATTCATCA
TACGAGTAGAGTGATTCAAGACAACTATAAAACGGTTTCCCATGGCGGAT
CCCTTCTACGGGTTTATCATTACAAGGATAAAAAGTATTGTTGCGAAGAC
GAGAGCCTCTTGAAAAAACGGCATGGAGATCAACTTCGGGAAAAATTCGA
TTCAGTTGTTGGTCTTTTAGACTTGTAG

SEQ ID NO: 2 (also listed in NCBI Ref Seq NP_{—}504545.2)

MPRITASKIVLLIALSFCITVIYHFPIATRSSKEYDEYGNEYENVASIES
DIKNVRRLLDEVPDPSQNRLQFLKLDEHAFAFSAYTDDRNGNMGYKYVRV
LMFITSQDNFSCEINGRKSTDVSLYEFSENHKMKWQMFILNCKLPDGIDF
NNVSSVKVIRSTTKQFVDVPIRYRIQDEKIITPDEYDYKMSICVPALFGN
GYDAKRIVEFIELNTLQGIEKIYIYTNQKELDGSMKKTLKYYSDNHKITL
IDYTLPFREDGVWYHGQLATVTDCLLRNTGITKYTFFNDFDEFFVPVIKS
RTLFETISGLFEDPTIGSQRTALKYINAKIKSAPYSLKNIVSEKRIETRF
TKCVVRPEMVFEQGIHHTSRVIQDNYKTVSHGGSLLRVYHYKDKKYCCED
ESLLKKRHGDQLREKFDSVVGLLDL

SEQ ID NO: 3 is the nucleic acid sequence coding for SEQ ID NO: 4: (also listed in NCBI Ref Seq XM_{—}001674213.1; coding for galactosyltransferase from Caenorhabditis briggsae)

ATGCCACGAA TAACGGCAAG CAAAATAGTG TTATTATCTG
TATTATCCTT ACTAACAGTT TTCTATCTGA ATACATTTTC
GTCTATTAAA ATTGAAAACG ATCTCGACGG GACTGATTAC
GACTTGGATT ACATAGAATC TGATATCAAA AAGACGCGTC
GATTACTCAA TGAAATCCCT GATCCATCTC AAAACCGAGT
TCAATTTTTT AAACTCGATG ATAATGGATA TGCATTCTCA
GCATATACAG ATAATAGGAA AGGAAATATG GGTCACAAAT
ATGTCAGAAT ATTAGTGTTC CTAACTAAAT TTGATGATTT
TTCTTGCGAA ATTAACTCGA AGAAATCCTA TGTTGTTACA
CTCTACGAGC TATCAGAAAA TCACAATATG AAGTGGAAAA
TGTATATTTT GAATTGTTTA CTTCCCGATG GAATCACTTT
CAACGATGTG AATTCTGTAA AAATATCTAG AAGTTCTTCA
AAACTTTCAG TCCAAATCCC GATCAGATAT AGAATTCAAG
ATGAGAAAAT GATGACTCCA GATGAATACG ATTATAAGTT
GTCGATTTGT GTTCCTGCAC TTTTTGGAAA CGTTTATTAT
CCAAGGAGGA TTATTGAATT TGTGGAACTA AACAGCTTGC
AAGACATCGA CAAAATCTAC ATCTACTACA ATCCTTTAGA
AATGACAGAT GAGGCCACAG AAAGGACTTT GAAGTTTTAT
TCCAATAATG GGAAAATCAA TTTAATAGAA TTCATTCTCC
CATTTTCTAC TCGAGATGTT TGGTATTATG GGCAATTGGC
CACCGTTACA GATTGTCTTC TCCGTAACAC TGGAATAACT
CAATACACAT TTTTCAATGA TTTGGATGAA TTTTTCGTGC
CAGTACTGGA CAACCAAACT CTCTCTGAAA CTGTGTCAGG
ATTATTTGAA AATCGAAAAA TTGCCTCTCA GAGAACGGCC
TTGAAATTTA TTAGTACAAA AATCAATCGA TCTCCTGTAA
CTCTCAATAA TATTGTGTCT TCTAAAAATT TTGAAACGAG
ATTCACAAAA TGCGTCGTAC GGCCGGAAAT GGTTTTTGAG
CAGGGCATTC ACCATACGAG TAGAGTAATA CAAGACGACT
ACGAAACCCC ATCCCATGAT GGATCACTTT TGCGTGTGTA
TCACTACAGA GAACCAAGAT ATTGCTGCGA AAACGAGAAT
CTTCTAAAAC AAAGATACGA TAAGAAGCTT CAAGAAGTTT
TTGATGCTGT AGTTCTTATA TTGCATGTCA CATTTGATGT
ATGGATATAT CACCTGAAAA ACACCCTCTA A

SEQ ID NO: 4 (also listed in NCBI Ref Seq XP_{—}001674265.1)

MPRITASKIV LLSVLSLLTV FYLNTFSSIK IENDLDGTDY
DLDYIESDIK KTRRLLNEIP DPSQNRVQFF KLDDNGYAFS
AYTDNRKGNM GHKYVRILVF LTKFDDFSCE INSKKSYVVT
LYELSENHNM KWKMYILNCL LPDGITFNDV NSVKISRSSS
KLSVQIPIRY RIQDEKMMTP DEYDYKLSIC VPALFGNVYY
PRRIIEFVEL NSLQDIDKIY IYYNPLEMTD EATERTLKFY
SNNGKINLIE FILPFSTRDV WYYGQLATVT DCLLRNTGIT
QYTFFNDLDE FFVPVLDNQT LSETVSGLFE NRKIASQRTA
LKFISTKINR SPVTLNNIVS SKNFETRFTK CVVRPEMVFE
QGIHHTSRVI QDDYETPSHD GSLLRVYHYR EPRYCCENEN
LLKQRYDKKL QEVFDAVVLI LHVTFDVWIY HLKNTL

SEQ ID NO: 5 is the nucleic acid sequence coding for SEQ ID NO: 6 (1428 nucleic acids) followed by a stop codon and further 68 nucleotides: (also listed in NCBI Ref Seq XM_{—}001629141.1; coding for galactosyltransferase from Nematostella vectensis)

ATGCGATGCT ATATTTACAA ATTGAGGTTG TCCGTTTGTC
TGTTTGTAGT GCTCTTCACA GCACTGCTTT TCATCACCTA
TTTAAACCAC TCAGAGCTTG AATCAGCAGA GAAAAGTAGC
GGAAAAAGGA AGACGCGACA TCGTAAACGA ACACGTTCAC
GCAAACAACA CGAGAGCCAT TTTCAGAAAG CTCGACTACA
AGAAAGAGAA CTAGTATTAA GATCTACAGC GCCACCAACA
TTACGAAGAG AAGTACAAGC GCATCGATTA GGGCAGATCC
GTGGCAAGAA CACGGACCAG GGGATAACTG GAAAGTTCAC
AGAGATCGCT AAAGACACGC ATATTTATTC AGCGTTTTAC
GACGATGCCA AGTCAAATCC ATTCATTCGT CTTATCATCC
TCTCGGGAAA ACACTACCAG CCTGGATTAT CTTGCCAATT
TTGCGAACCT TTGTCCGCCA GTTGTAGTTT TGCGGACTCT
AAAGCTGAAT ACTACACGAC CAACGAGAAC CATGGGAGAG
TATTTGGCGG GTTCATTGCG AGTTGCCTCG TGCCTGATGG
ATTCAATGCA GTGCCATTGT TTGTTGACAT AACGGCCGAT
GTTAAGGGGG AGAAAAGCAA GGCACGGGTA CCTGTGGTGT
CTAATGCACA TCTCTACTAC CCTATTAAAT ACGCAATCTG
CGTCCCACCC CTCCGATCAG AGAAACTAAC AGCGAAAAGA
CTCATAGAGT TTGTCGAGCT AACCAAACTT TTAGGCGCTA
ACCATTTTAC TTTTTATGAC TTCAAAACGG ACCCGGAAGT
CAATAACGTT TTAAGATATT ACCAGGAGAC ACAAGTAGCA
AATGTTCTGC CATGGAATCT ACCTTCAAAT TTGGTATCCA
GGCCGAACGA TATTTGGTAC TTTGGTCAGG TTTTGGCTAT
TCTAGATTGC TTGTATCGCT ACAAGAACAG GGCAAAATTT
GTAGCCTTCA ATGACGTAGA TGAGTTTATC GTTCCGCTAA
GGAACAGCTC GATAGTGGAA ATACTAAACG CGTTTCACCG
GCCATACCAC TGTGGACATT GCTTTCAGAG CGTGGTGTTC
AGCTCAAACG CGAGATTTCC CAGGCAAAAA AGCGAGTTAG
TTTCTCAGCG GTTCTTCCAC AGGACCCAGG AAACCATCCC
TCTCCTCTCG AAATGCATTG TGGATCCTTT GAGAGTGTTC
GAGATGGGGA TTCACCACAT AAGCAAGGCT ACAGGTCTGC
GGTATTCCGT CAACTCAGTA CACGAGAGTG ACGCGGTTAT
CTTCCATTAC AGGACTTGCA CTACGTCATT TGGTATACGT
CATCAGTGCA TGAACCTAGT GCATGATGGG ACCATGGCCA
AATATGGAAA ACGACTTCAG AAAATGTTTA GAAAGGTTGT
AAATGATTTA AAACTTTTGG CACCAACGTA GCTATTTCGT
AACACTTCAC ACTTTCATTG TTATAACAGA ATACAGAATA
AATTAATGAT TGTTGTGCC

SEQ ID NO: 6 (also listed in NCBI Ref Seq XP_{—}001629191)

MRCYIYKLRL SVCLFVVLFT ALLFITYLNH SELESAEKSS
GKRKTRHRKR TRSRKQHESH FQKARLQERE LVLRSTAPPT
LRREVQAHRL GQIRGKNTDQ GITGKFTEIA KDTHIYSAFY
DDAKSNPFIR LIILSGKHYQ PGLSCQFCEP LSASCSFADS
KAEYYTTNEN HGRVFGGFIA SCLVPDGFNA VPLFVDITAD
VKGEKSKARV PVVSNAHLYY PIKYAICVPP LRSEKLTAKR
LIEFVELTKL LGANHFTFYD FKTDPEVNNV LRYYQETQVA
NVLPWNLPSN LVSRPNDIWY FGQVLAILDC LYRYKNRAKF
VAFNDVDEFI VPLRNSSIVE ILNAFHRPYH CGHCFQSVVF
SSNARFPRQK SELVSQRFFH RTQETIPLLS KCIVDPLRVF
EMGIHHISKA TGLRYSVNSV HESDAVIFHY RTCTTSFGIR
HQCMNLVHDG TMAKYGKRLQ KMFRKVVNDL KLLAPT

SEQ ID NO: 7 is the nucleic acid sequence coding for SEQ ID NO: 8: (also listed in NCBI Ref Seq XM_{—}002189335, coding for galactosyltransferase from Taeniopygia guttata)

ATGACTGTAA CTTTAATGCT TGTGGTTTCT TATCTGAGAT
TACAGAGACT TTCTCATCAG CCAAAAGTAA TTCAAGAAAG
TAGAAGATGT AGAGGGAAAA TTGCCCTTAG CACAATAACA
GCATTGGAAG GTAACAAAAC TGATATTATA TCCCCATACT
TTGATGACAG AGAAAACAAA ATCACTCGTC TGATTGGGAT
TGTTCACCAT AAAGATGTAA AACAACTGTT CTGCTGGTTC
TGCTGTCAAG CCAATGGAAA GATATATGTA TCAAAAGCAG
AAATAGATGT TCACTCGGAT AGATTTGGAT TCCCTTATGG
TGCAGCAGAT ATAATTTGTT TGGAACCTGA AAACTGTGAT
CCAACACATG TATCAATTCA TCAGTCTCCA TATGGAAATA
TTGACCAGCT GCCGAGGTTT GAAATTAAAA ATCGCAGGCC
TGAGACCTTT TCTGTTGACT TCACCGTGTG CATTTCTGCC
ATGTTTGGAA ACTACAACAA TGTCTTGCAG TTTGTACAGA
GTATGGAAAT GTATAAGATT CTTGGAGTAC AGAAAGTGGT
GATCTATAAG AACAACTGCA GCCATCTGAT GGAGAAAGTC
TTGAAATTTT ATATAGAAGA AGGAACTGTT GAGGTAATTC
CCTGGCCAAT AGACTCACAC CTCAGGGTTT CTTCTAAATG
GCGCTTCATG GAAGACGGGA CACACATTGG CTACTATGGA
CAAATCACAG CTCTAAATGA CTGTATATAC CGCAACATGG
AAAGGACCAA GTTTGTGGTC CTTAATGACG CTGATGAAAT
AATTCTTCCC CTTAAACACC CAGACTGGAA AACAATGATG
AACAGTCTTC AGGAGCAAAA CCCAGGGACT AGTGTTTTCC
TTTTTGAGAA CCATATCTTC CCAGAAACTG TATTTTCTCC
CATGTTCAAC ATTTCATCTT GGAATACTGT GCCAGGTGTT
AACATATTGC AGCATGTGTA CAGAGAGCCT GACAGGAAAC
ATGTAATCAA TCCCAGGAAA ATGATAGTTG ATCCACGAAA
GGTGATTCAG ACTTCAGTCC ATTCTGTCCT ACGTGCTTAT
GGGAAGAGCG TGAATGTTCC CATGGAAGTT GCCCTCATTT
ATCACTGTCG GAAGGCCCTT CAAGGAAACC TTCCCAGAGA
ATCTCTCATC AGGGATACAA CACTGTGGAG ATATAACTCA
TCATTAATCA TGAATGTTAA CAAGGTTCTA TCTCAAACCA
TGCTGCAAAC TCAAAATTGA

SEQ ID NO: 8 (also listed in NCBI Ref Seq XP_{—}002189371)

MTVTLMLVVS YLRLQRLSHQ PKVIQESRRC RGKIALSTIT
ALEGNKTDII SPYFDDRENK ITRLIGIVHH KDVKQLFCWF
CCQANGKIYV SKAEIDVHSD RFGFPYGAAD IICLEPENCD
PTHVSIHQSP YGNIDQLPRF EIKNRRPETF SVDFTVCISA
MFGNYNNVLQ FVQSMEMYKI LGVQKVVIYK NNCSHLMEKV
LKFYIEEGTV EVIPWPIDSH LRVSSKWRFM EDGTHIGYYG
QITALNDCIY RNMERTKFVV LNDADEIILP LKHPDWKTMM
NSLQEQNPGT SVFLFENHIF PETVFSPMFN ISSWNTVPGV
NILQHVYREP DRKHVINPRK MIVDPRKVIQ TSVHSVLRAY
GKSVNVPMEV ALIYHCRKAL QGNLPRESLI RDTTLWRYNS
SLIMNVNKVL SQTMLQTQN

SEQ ID NO: 9 is the nucleic acid sequence coding for SEQ ID NO: 10: (also listed in NCBI Ref Seq XM_{—}626032, coding for galactosyltransferase from Cryptosporidium parvum)

ATGCAAAGTA AAGTCATTTT TAGGATCTTG GTATTGATCA
TTTCGGTGAT TGGATCCTTA TACTCAATAA TTCAATTAAT
GCTAAAGGAG CTATCAAGTA ACAAAAATAT TCAAGAGGTT
AGTCATTCAA GGAGGCTAAT AAGTGAACCT TACAGTGAAA
GTATTAATGA ACAAAATGAT CAAGATTGGA AAGAACTAAA
GCTAATAATT CCAAATCATT CTCAAATTAA CCAGCAGGAA
AAAAATGGTA ATTTGATTGA GTTTAAAGTT TATATATACT
CAGCATATTA TGATTGGAGA ATAGATAGGA TACGAATAAA
TTCACTTATC CCATCGAATT TTTATGATCG AATAGAAATG
GAATGTGCAA TAATCTTGGA CAAAAATATT TACACAGGAA
CTATTAAAAA AGTGATTCAT AAGGAGCACC ATAATAAAGA
ATATGTATCA TCGACTTTAC TCTGCGAAAT TGCAAAAAAT
GAAATTAAAT TTGAGGATAT TTCAAGGAAA GTTTTGATAA
CAATTTTGGA AAATGGAAAC AGCACAAATA AATCAGAAAT
ATGGATAACT CTAAAAAAAA TTCCAAAAAA TAGCTCTAAT
AATCATGAGC TGACTGTTTG TGTGAGACCT TGGTGGGGAG
AGCCAATAAA GAATGGAAAC TTGGGAAATA AACAAAAATT
TAACAATTCA GGGTTAATGC TTGAATTTAT TAATTCATAT
TTATTCTTAG GAGCAAATAA ATTTTATTTA TATCAAAATT
ACTTGGACAT TGACGAAGAT GTAAGAAATA TAATAAATTA
TTATTCTAAT ATCAAAAATG TTTTGGAAAT TATTCCATAC
TCATTACCAA TAATTCCATT TAAACAAGTT TGGGATTTCG
CACAAACAAC AATGATACAG GACTGCCTAC TAAGAAATAT
TGGAAAAACA AAATACTTGT TATTCGTAGA TACCGATGAA
TTTGTATTTC CAAACTTGAA AAATTATAAC TTAATGGATT
TTTTAAATTT ATTAGAAGCC AACAATCCTT ATTATAAAAA
CAAAGTCGGG GCAATGTGGA TTCCAATGTA TTTTCATTTT
TTAGAGTGGG AATCTGATAA AAATAATTTG AAGAAATATT
CAACAATTGA GAAAAAAATT AAGAAAAAGA TGGCAAATAT
TGAGTTTGTT CTATATCGTA AAACATGTAG AATGTTAAGT
TCTGGAACAA AAAAAAGTGA CAAGACGAGA AGAAAAGTTA
TTATTAGACC TGAAAGAGTT TTGTATATGG GTATACATGA
AACAGAAGAG ATGCTAAGCA AAAAATTTCA TTTCATTAGA
GCTCCTGTAA TTAATGTGGG TGGAGGAAAC GAACTAAGTA
TATATTTACA TCATTATAGA AAAGCAAAAG GTATTGTAAA
CAATGATCCC AAACAAAGAG AACTTGTGAA TATGTATTTA
GAAAATGTTT GTTCAGATAA GCTGTTAGAT TCAGGGGGAG
ATTCCATTCA AGATGGAGTA ATTGTCGACA ATACTGTTTG
GGAGATATTT GGAACACACT TATACCAGAT AATTTTTGAG
CATATTAAAG AAATCCAAGA TATGTACACA AATAAGGAAA
TAATTAATGG AAATAAAAAT TTAAGTGTTG AAGAATTACA
TAATTAA

SEQ ID NO: 10 (also listed in NCBI Ref Seq XP_{—}626032)

MQSKVIFRIL VLIISVIGSL YSIIQLMLKE LSSNKNIQEV
SHSRRLISEP YSESINEQND QDWKELKLII PNHSQINQQE
KNGNLIEFKV YIYSAYYDWR IDRIRINSLI PSNFYDRIEM
ECAIILDKNI YTGTIKKVIH KEHHNKEYVS STLLCEIAKN
EIKFEDISRK VLITILENGN STNKSEIWIT LKKIPKNSSN
NHELTVCVRP WWGEPIKNGN LGNKQKFNNS GLMLEFINSY
LFLGANKFYL YQNYLDIDED VRNIINYYSN IKNVLEIIPY
SLPIIPFKQV WDFAQTTMIQ DCLLRNIGKT KYLLFVDTDE
FVFPNLKNYN LMDFLNLLEA NNPYYKNKVG AMWIPMYFHF
LEWESDKNNL KKYSTIEKKI KKKMANIEFV LYRKTCRMLS
SGTKKSDKTR RKVIIRPERV LYMGIHETEE MLSKKFHFIR
APVINVGGGN ELSIYLHHYR KAKGIVNNDP KQRELVNMYL
ENVCSDKLLD SGGDSIQDGV IVDNTVWEIF GTHLYQIIFE
HIKEIQDMYT NKEIINGNKN LSVEELHN

The term “nucleic acid encoding a polypeptide” as it is used in the context of the present invention is meant to include allelic variations and redundancies in the genetic code.

The term “% (percent) identity” as known to the skilled artisan and used herein indicates the degree of relatedness among two or more nucleic acid molecules that is determined by agreement among the sequences. The percentage of “identity” is the result of the percentage of identical regions in two or more sequences while taking into consideration the gaps and other sequence peculiarities.

The identity of related nucleic acid molecules can be determined with the assistance of known methods. In general, special computer programs are employed that use algorithms adapted to accommodate the specific needs of this task. Preferred methods for determining identity begin with the generation of the largest degree of identity among the sequences to be compared. Preferred computer programs for determining the identity among two nucleic acid sequences comprise, but are not limited to, BLASTN (Altschul et al., J. Mol. Biol., 215, 403-410,1990) and LALIGN (Huang and Miller, Adv. Appl. Math., 12, 337-357, 1991). The BLAST programs can be obtained from the National Center for Biotechnology Information (NCBI) and from other sources (BLAST handbook, Altschul et al., NCB NLM NIH Bethesda, Md. 20894).

The nucleic acid molecules according to the invention may be prepared synthetically by methods well-known to the skilled person, but also may be isolated from suitable DNA libraries and other publicly available sources of nucleic acids and subsequently may optionally be mutated. The preparation of such libraries or mutations is well-known to the person skilled in the art.

In a preferred embodiment, the nucleic acid molecules of the invention are cDNA, genomic DNA, synthetic DNA, RNA or PNA, either double-stranded or single-stranded (i.e. either a sense or an anti-sense strand). The nucleic acid molecules and fragments thereof, which are encompassed within the scope of the invention, may be produced by, for example, polymerase chain reaction (PCR) or generated synthetically using DNA synthesis or by reverse transcription using mRNA from Caenorhabditis elegans, Caenorhabditis briggsae, Nematostella vectensis, Taeniopygia guttata or Cryptosporidium parvum.

In some instances the present invention also provides novel nucleic acids encoding the polypeptides of the present invention characterized in that they have the ability to hybridize to a specifically referenced nucleic acid sequence, preferably under stringent conditions. Next to common and/or standard protocols in the prior art for determining the ability to hybridize to a specifically referenced nucleic acid sequence under stringent conditions (e.g. Sambrook and Russell, Molecular cloning: A laboratory manual (3 volumes), 2001), it is preferred to analyze and determine the ability to hybridize to a specifically referenced nucleic acid sequence under stringent conditions by comparing the nucleotide sequences, which may be found in gene databases (e.g. http://www.-ncbi.nlm.nih.gov/entrez/query.fcgi?db=nucleotide) with alignment tools, such as e.g. the above-mentioned BLASTN (Altschul et al., J. Mol. Biol., 215, 403-410,1990) and LALIGN alignment tools.

Most preferably the ability of a nucleic acid of the present invention to hybridize to a nucleic acid, e.g. those listed in any of SEQ ID NOs 1, 3, 5, 7 and/or 9, is confirmed in a Southern blot assay under the following conditions: 6× sodium chloride/sodium citrate (SSC) at 45° C. followed by a wash in 0.2×SSC, 0.1% SDS at 65° C.

The nucleic acid of the present invention is preferably operably linked to a promoter that governs expression in suitable vectors and/or host cells producing the polypeptides of the present invention in vitro or in vivo.

Suitable promoters for operable linkage to the isolated and purified nucleic acid are known in the art. In a preferred embodiment the nucleic acid of the present invention is one that is operably linked to a promoter selected from the group consisting of the Pichia pastoris AOX1 or GAP promoter (see for example Pichia Expression Kit Instruction Manual, Invitrogen Corporation, Carlsbad, Calif.), the Saccharomyces cerevisiae GAL1, ADH1, ADH2, MET25, GPD or TEF promoter (see for example Methods in Enzymology, 350, 248, 2002), the Baculovirus polyhedrin p10 or ie1 promoter (see for example Bac-to-Bac Expression Kit Handbook, Invitrogen Corporation, Carlsbad, Calif., and Novagen Insect Cell Expression Manual, Merck Chemicals Ltd., Nottingham, UK), the E. coli T7, araBAD, rhaP BAD, tetA, lac, trc, tac or pL promoter (see Applied Microbiology and Biotechnology, 72, 211, 2006), the plant CaMV35S, ocs, nos, Adh-1, Tet promoters (see e.g. Lau and Sun, Biotechnol Adv. 27, 1015-1022, 2009) or inducible promoters for mammalian cells as described in Sambrook and Russell (2001).

Preferably, the isolated and purified nucleic acid is in the form of a recombinant vector, such as an episomal or viral vector. The selection of a suitable vector and expression control sequences as well as vector construction are within the ordinary skill in the art. Preferably, the viral vector is a baculovirus vector (see for example Bac-to-Bac Expression Kit Handbook, Invitrogen Corporation, Carlsbad, Calif.). Vector construction, including the operable linkage of a coding sequence with a promoter and other expression control sequences, is within the ordinary skill in the art.

Hence and in a further aspect, the present invention relates to a recombinant vector, comprising a nucleic acid of the invention.

A further aspect of the present invention is directed to a host cell comprising a nucleic acid and/or a vector of the invention and preferably producing polypeptides of the invention. Preferred host cells for producing the polypeptide of the invention are selected from the group consisting of yeast cells, preferably Saccharomyces cerevisiae (see for example Methods in Enzmology, 350, 248, 2002), Pichia pastoris cells (see for example Pichia Expression Kit Instruction Manual, Invitrogen Corporation, Carlsbad, Calif.), E. coli cells (BL21(DE3), K-12 and derivatives) (see for example Applied Microbiology and Biotechnology, 72, 211, 2006), plant cells, preferably Nicotiana tabacum or Physcomitrella patens (see e.g. Lau and Sun, Biotechnol Adv. 27, 1015-1022, 2009), NIH-3T3 mammalian cells (see for example Sambrook and Russell, 2001) and insect cells, preferably sf9 insect cells (see for example Bac-to-Bac Expression Kit Handbook, Invitrogen Corporation, Carlsbad, Calif.)

Another important aspect of the invention is directed to an isolated and purified polypeptide selected from the group consisting of
(a) polypeptides having an amino acid sequence selected from the group consisting of SEQ ID NOs: 2, 4, 6, 8 and 10, preferably SEQ ID NO: 2,
(b) polypeptides encoded by a nucleic acid of the present invention,
(c) polypeptides having an amino acid sequence identity of at least 25, 30 or 40%, preferably at least 50 or 60%, more preferably at least 70 or 80%, most preferably at least 90 or 95% with the polypeptides of (a) and/or (b),
(d) a fragment and/or functional derivative of (a), (b) or (c).

The identity of related amino acid molecules can be determined with the assistance of known methods. In general, special computer programs are employed that use algorithms adapted to accommodate the specific needs of this task. Preferred methods for determining identity begin with the generation of the largest degree of identity among the sequences to be compared. Preferred computer programs for determining the identity among two amino acid sequences comprise, but are not limited to, TBLASTN, BLASTP, BLASTX or TBLASTX (Altschul et al., J. Mol. Biol., 215, 403-410, 1990). The BLAST programs can be obtained from the National Center for Biotechnology Information (NCBI) and from other sources (BLAST handbook, Altschul et al., NCB NLM NIH Bethesda, Md. 20894).

Preferably, said polypeptides are encoded by an above-mentioned nucleic acid of the invention.

In a preferred embodiment, the polypeptide, fragment and/or derivative of the invention is functional, i.e. has enzymatic galactosyltransferase activity, preferably an enzymatic β-1,4-galactosyltransferase activity, more preferably an enzymatic β-1,4-galactosyltransferase activity, preferably with L-fucoside-, more preferably with α-L-fucoside-, more preferably with Fuc-α-1,6-GlcNAc- and most preferably with GnGnF⁶- (nomenclature according to Schachter, Biochem. Cell. Biol. 64(3), 163-181, 1986) containing poly/oligosaccharides or glycoconjugates as acceptor substrates.

For example, a preferred assay for determining the functionality, i.e. enzymatic activity, of the polypeptides, fragments and derivatives thereof according to the present invention is provided in example 4 below.

The term “functional derivative” of a polypeptide of the present invention is meant to include any polypeptide or fragment thereof that has been chemically or genetically modified in its amino acid sequence, e.g. by addition, substitution and/or deletion of amino acid residue(s) and/or has been chemically modified in at least one of its atoms and/or functional chemical groups, e.g. by additions, deletions, rearrangement, oxidation, reduction, etc. as long as the derivative still has at least one of the above enzymatic activities to a measurable extent, e.g. of at least about 1 to 10% of the original unmodified polypeptide.

In this context a functional fragment of the invention is one that forms part of a polypeptide or derivative of the invention and still has at least one of the above enzymatic activities in a measurable extent, e.g. of at least about 1 to 10% of the complete protein.

The term “isolated and purified polypeptide” as used herein refers to a polypeptide or a peptide fragment which either has no naturally-occurring counterpart (e.g., a peptide-mimetic), or has been separated or purified from components which naturally accompany it, e.g. in Caenorhabditis elegans tissue or a fraction thereof. Preferably, a polypeptide is considered “isolated and purified” when it makes up for at least 60% (w/w) of a dry preparation, thus being free from most naturally-occurring polypeptides and/or organic molecules with which it is naturally associated. Preferably, a polypeptide of the invention makes up for at least 80%, more preferably at 90%, and most preferably at least 99% (w/w) of a dry preparation. More preferred are polypeptides according to the invention that make up for at least 80%, more preferably at least 90%, and most preferably at least 99% (w/w) of a dry polypeptide preparation. Chemically synthesized polypeptides are by nature “isolated and purified” within the above context.

An isolated polypeptide of the invention may be obtained, for example, by extraction from a natural source, e.g. Caenorhabditis elegans, Caenorhabditis briggsae, Nematostella vectensis, Taeniopygia guttata or Cryptosporidium parvum; by expression of a recombinant nucleic acid encoding the polypeptide in a host, preferably a heterologous host; or by chemical synthesis. A polypeptide that is produced in a cellular system being different from the source from which it naturally originates is “isolated and purified”, because it is separated from components which naturally accompany it. The extent of isolation and/or purity can be measured by any appropriate method, e.g., column chromatography, polyacrylamide gel electrophoresis, HPLC analysis, NMR spectroscopy, gas liquid chromatography, or mass spectrometry.

Furthermore, in one aspect the present invention relates to antibodies, functional fragments and functional derivatives thereof that specifically bind a polypeptide of the invention. These are routinely available by hybridoma technology (Kohler and Milstein, Nature, 256, 495-497, 1975), antibody phage display (Winter et al., Annu. Rev. Immunol. 12, 433-455, 1994), ribosome display (Schaffitzel et al., J. Immunol. Methods, 231, 119-135, 1999) and iterative colony filter screening (Giovannoni et al., Nucleic Acids Res. 29, E27, 2001) once the target antigen is available. Typical proteases for fragmenting antibodies into functional products are well-known. Other fragmentation techniques can be used as well as long as the resulting fragment has a specific high affinity and, preferably a dissociation constant in the micromolar to picomolar range.

A very convenient antibody fragment for targeting applications is the single-chain Fv fragment, in which a variable heavy and a variable light domain are joined together by a polypeptide linker. Other antibody fragments for identifying the polypeptide of the present invention include Fab fragments, Fab₂ fragments, miniantibodies (also called small immune proteins), tandem scFv-scFv fusions as well as scFv fusions with suitable domains (e.g. with the Fc portion of an immunoglobulin). For a review on certain antibody formats, see Holliger and Hudson, Biotechnol., 23(9), 1126-36, 2005.

The term “functional derivative” of an antibody for use in the present invention is meant to include any antibody or fragment thereof that has been chemically or genetically modified in its amino acid sequence, e.g. by addition, substitution and/or deletion of amino acid residue(s) and/or has been chemically modified in at least one of its atoms and/or functional chemical groups, e.g. by additions, deletions, rearrangement, oxidation, reduction, etc. as long as the derivative has substantially the same binding affinity as to its original antigen and, preferably, has a dissociation constant in the micro-, nano- or picomolar range.

In a preferred embodiment, the antibody, fragment or functional derivative thereof for use in the invention is one that is selected from the group consisting of polyclonal antibodies, monoclonal antibodies, chimeric antibodies, humanized antibodies, CDR-grafted antibodies, Fv-fragments, Fab-fragments and Fab₂-fragments and antibody-like binding proteins, e.g. affilines, anticalines and aptamers.

For a review of antibody-like binding proteins see Binz et al. on engineering binding proteins from non-immunoglobulin domains in Nature Biotechnol., 23(10), 1257-1268, 2005. The term “aptamer” describes nucleic acids that bind to a polypeptide with high affinity. Aptamers can be isolated from a large pool of different single-stranded RNA molecules by selection methods such as SELEX (see, e.g., Jayasena, Clin. Chem., 45, 1628-1650, 1999; Klug and Famulok, M. Mol. Biol. Rep., 20, 97-107, 1994; U.S. Pat. No. 5,582,981). Aptamers can also be synthesized and selected in their mirror form, for example, as the L-ribonucleotide (Nolte et al., Nat. Biotechnol., 14, 1116-1119, 1996; Klussmann et al., Nat. Biotechnol., 14, 1112-1115, 1996). Forms isolated in this way have the advantage that they are not degraded by naturally occurring ribonucleases and, therefore, have a greater stability.

Another antibody-like binding protein and alternative to classical antibodies are the so-called “protein scaffolds”, for example, anticalines, that are based on lipocaline (Beste et al., Proc. Natl. Acad. Sci. USA, 96, 1898-1903, 1999). The natural ligand binding sites of lipocalines, for example, of the retinol-binding protein or bilin-binding protein, can be changed, for example, by employing a “combinatorial protein design” approach, and in such a way that they bind selected haptens (Skerra, Biochem. Biophys. Acta, 1482, pp. 337-350, 2000). For other protein scaffolds it is also known that they are alternatives for antibodies (Skerra, J. Mol. Recognition, 13, 167-287, 2000; Hey, Trends in Biotechnology, 23, 514-522, 2005).

In summary, the term functional antibody derivative is meant to include the above protein-derived alternatives for antibodies, i.e. antibody-like binding proteins, e.g. affilines, anticalines and aptamers, that specifically recognize a polypeptide, fragment or derivative threof.

A further aspect relates to a hybridoma cell line, expressing a monoclonal antibody according to the invention.

The nucleic acids, vectors, host cells, polypeptides and antibodies of the present invention have a number of new applications.

In one aspect the present invention relates to the use of a polypeptide, a cell extract comprising a polypeptide of the invention, preferably a nematode extract, more preferably an extract of Caenrhabditis elegans, Caenorhabditis briggsae, Nematostella vectensis, Taeniopygia guttata or Cryptosporidium parvum, and/or a host cell of the present invention for producing galactoside-containing oligo/polysaccharides and/or glycoconjugates, preferably galactosyl-fucoside-containing oligo/polysaccharides and glycoconjugates, more preferably D-galactopyranosyl-β-1,4-L-fucopyranosyl-α-1,6-GlcNAc-containing oligo/polysaccharides and glycoconjugates, most preferably GnGnF⁶Gal- or MMF⁶Gal-containing oligo/polysaccharides and glycoconjugates.

It is understood that the term glycoconjugate, as used herein is non-limiting with respect to the nature of the non-sugar component. Preferably the non-sugar component of the glycoconjugate is a poly/oligopeptide.

The enzymatic synthesis of galactosyl-fucosyl-specific oligosaccharides and glycoconjugates is highly specific, controlled and environment-friendly and the products can serve as highly parasite-specific (this epitope is only known to also exist in octopus [Zhang et al., Glycobiology, 7, 1153-1158, 1997], squid [Takahashi et al., Eur. J. Biochem., 270, 2627-2632, 2003] and limpets [Wuhrer et al., Biochem. J., 378, 625-632, 2004]) vaccine components for the treatment and prevention of parasitic, preferably nematode and apicomplexa infections in a subject, such as a human or other mammal, in need thereof.

Exemplary and preferred galactosyl-fucosyl-specific oligosaccharides and glycoconjugates are selected from the group consisting of N-linked glycans, N-glycoproteins, glycolipids and lipid-linked oligosaccharides (LOS). The term “glycoconjugate” as used herein, is meant to include any type of conjugate, preferably but not necessarily a covalently bonded one, for example bonded by a covalent linker, of an oligosaccharide- and a non-saccharide component, e.g. a polypeptide or any other type of organic or inorganic carrier that is physiologically acceptable and might even have a desired physiological function, e.g. as an immune stimulating adjuvant, imparting nematode toxicity, etc.

For example, raw extracts of Caenorhabditis elegans, Caenorhabditis briggsae, Nematostella vectensis, Taeniopygia guttata or Cryptosporidium parvum or recombinant insect cells producing a polypeptide of the invention can produce Gal-Fuc-containing conjugates, e.g. free Gal-Fuc glycans, Gal-Fuc-peptides, Gal-Fuc-polypeptides, Gal-Fuc-folded proteins. Alpha-1,6-linked fucosides are strongly preferred over alpha-1,3-linked fucosides.

Another aspect of the present invention is directed to a method for producing galactosyl-fucosyl derivatives, comprising the following steps:
(i) providing at least one polypeptide of the invention,
(ii) providing at least one fucosylated acceptor substrate,
(iii) incubating (i) and (ii) in the presence of at least one suitable divalent metal cation cofactor, preferably selected from manganese (II), cobalt (II) and/or iron (II) ions, more preferably manganese (II), and at least one activated sugar substrate, preferably uridine diphosphate (UDP)-galactose under conditions suitable for enzymatic activity of the polypeptide of the invention,
(iv) optionally isolating the galactosyl-fucose derivatives.

The polypeptide of the invention may be provided as an isolated polypeptide, in dry or soluble form, in a buffer, a host cell, a cell extract or any other system that will sustain its enzymatic activity and allow access to its substrate and activated sugar substrate. The fucosylated acceptor substrate is any kind of fucosyl-containing substrate, optionally in isolated form or as a component of a system that can be enzymatically modified by the polypeptide of the invention. The activated sugar substrate is preferably UDP-galactose but can also be any other type of activated, preferably phosphate-activated galactosyl derivative that can be transferred to a fucosylated acceptor substrate. The method of the invention preferably leads to galactopyranosyl-β-1,4-L-fucopyranosyl-derivatives, more preferably D-galactopyranosyl-β-1,4-L-fucopyranosyl-α-1,6-βGlcNAc (Gal-Fuc) derivatives.

The polypeptides of the present invention have a broad substrate specificity as long as the substrate features a suitable fucosyl-moiety. Galactosyl-transferase activity was demonstrated for substrates such as, e.g. fucosyl-saccharides, fucosyl-peptides, fucosyl-polypeptides and even complex and folded fucosyl-polypeptides. For example, galactosyl-transferase activity was demonstrated for human IgG1, a glycoprotein having GnGnF⁶ carbohydrate structures as prevalent epitopes. These IgG1 glycans are known to be accessible for PNGaseF digest. Glycosylation of human IgG1 was demonstrated with the crude sf9 insect cell extract containing the core galactosyltransferase of Caenorhabditis elegans. Incubation of human IgG1 with radioactively labelled UDP-Gal in the presence of enzyme extract from Caenorhabditis elegans led to substrate galactosylation. In addition, galactosylation was demonstrated on remodelled human transferrin carrying GnGnF⁶ carbohydrate structures as prevalent epitopes. For this purpose human apotransferrin was sequentially treated with sialidase (lskratsch et al, Anal. Biochem., 368, 133-146, 2009), β1,4-galactosidase from Aspergillus oryzae and recombinant Anopheles core α1,6-FucT expressed in Pichia pastoris to produce a glycoprotein having GnGnF⁶ carbohydrate structures as prevalent epitopes. Incubation with a crude sf9 insect cell extract containing the core galactosyltransferase of Caenorhabditis elegans led to galactosylation which was monitored by dot blotting with the fucose-specific Aleuria aurantia lectin and by MALDI-TOF MS of tryptic peptides of the various neoglycoforms.

It has very recently been shown that the serum content of core fucosylated alpha feto-protein (AFP) is highly specific for hepatocellular carcinomas (HCC), because benign liver diseases such as chronic hepatitis and liver cirrhosis do not lead to core-fucosylated AFP in mammals, in particular humans (see Tateno et al., Glycobiology, 19(5), 527-536. 2009).

Therefore, in a further aspect the polypeptides of the invention, host cells comprising polypeptides of the invention and/or cell extracts of Caenorhabditis elegans, Caenorhabditis briggsae, Nematostella vectensis, Taeniopygia guttata and/or Cryptosporidium parvum can be used for covalently binding galactosyl compounds to core-fucosylated alpha-fetoprotein (AFP), preferably for detecting and/or quantifying hepatocellular carcinoma (HCC) cells, preferably by selectively labelling core-fucosylated alpha-fetoprotein (AFP) from the blood of HCC patients, because core-fucosylated AFP is selectively suitable as an acceptor substrate for the polypeptides of the present invention.

Hence, the present invention relates to polypeptides of the invention, host cells comprising polypeptides of the invention and/or cell extracts of Caenorhabditis elegans, Caenorhabditis briggsae, Nematostella vectensis, Taeniopygia guttata and/or Cryptosporidium parvum for preparing diagnostic means for detecting core-fucosylated AFP, i.e. for detecting and/or quantifying hepatocellular carcinoma (HCC) cells.

Also, the polypeptides of the invention, host cells comprising polypeptides of the invention and/or cell extracts of Caenorhabditis elegans, Caenorhabditis briggsae, Nematostella vectensis, Taeniopygia guttata and/or Cryptosporidium parvum are useful for preparing diagnostic means for detecting further core-fucosylated marker glycoproteins whose appearance correlates with other types of carcinoma cells.

In a preferred embodiment, the invention relates to a method of diagnosis, comprising the following steps:

(i) providing blood or a fraction thereof, that comprises AFP, preferably serum,

(ii) incubating said blood or said fraction thereof with (a) a polypeptide of the invention, a host cell of the invention and/or cell extracts of Caenorhabditis elegans, Caenorhabditis briggsae, Nematostella vectensis, Taeniopygia guttata and/or Cryptosporidium parvum and (b) an activated galactosyl derivative, preferably a labelled galactosyl derivative, preferably labelled UDP-galactose, under conditions that allow for the galactosyltransfer of activated galactose to core-fucosylated AFP (AFP-L3),

(iii) and detecting the galactose-labelled and hence core-fucosylated AFP (AFP-L3).

Labels for activated galactosyl derivatives for practicing the above method are selected from the group consisting of isotopes e.g. ¹⁴C, chemical modifications e.g. halogen substitutions and other selectively detectable modifications e.g. biotin, azide etc. Preferably, all of the steps (i) to (iii) are performed outside the living body, i.e. in vitro.

A further aspect of the invention is directed to the use of antibodies specifically binding a polypeptide of the invention, preferably a polypeptide having a sequence selected from any of SEQ ID NOs: 2, 4, 6, 8 and/or 10, for identifying and/or quantifying nematodes and apicomplexa, preferably Caenorhabditis elegans, Caenorhabditis briggsae, and Cryptosporidium parvum, respectively, in a sample of interest, for example a human or mammalian sample, preferably in a cell fraction or extract sample. The design and development of typical antibody assays, e.g. ELISAs, is within the ordinary skill in the art and need not be further elaborated.

The invention has been described with the emphasis upon preferred embodiments and illustrative examples. However, it will be obvious to those of ordinary skill in the art that variations of the preferred embodiments may be used and that it is intended that the invention may be practiced otherwise than as specifically described herein. Moreover, as the foregoing examples are included for purely illustrative purposes, they should not be constructed to limit the scope of the invention in any respect. Accordingly, this invention includes all modifications encompassed within the spirit and scope of the invention as defined by the claims appended hereto.

### FIGURES

FIG. 1 is an anti-FLAG immunoblotting of baculovirus-infected sf9 whole cell extracts. Different clones of baculoviruses containing empty vector control (e.v.), N-terminally FLAG-tagged M03F8.4 (FLAG-GalT) and untagged M03F8.4 (GalT). Loading ca. 150 kcells/slot, SDS-PAGE 12%, α-FLAG (1:2000, SIGMA), α-mouse-HRP (1:2000, Santa Cruz Biotechnology), ECL (Pierce, 2 s exposure).

FIG. 2 is an SDS-PAGE analysis of baculovirus-infected sf9 whole cell extracts. Different clones of baculoviruses containing empty vector control (e.v.), N-terminally FLAG-tagged M03F8.4 (FLAG-GalT) and untagged M03F8.4 (GalT). Loading ca. 150 kcells/slot, SDS-PAGE 12%, detection by silver staining. (Protein is expressed in low amounts, not detectable by silver staining with respect to the empty vector construct in crude extracts.)

FIG. 3 is a column chart showing the galactosylation turnover of a GnGnF⁶ acceptor substrate (dabsyl-GEN[GnGnF⁶]R) in the presence of Mn²⁺, Mg²⁺ and EDTA demonstrating metal ion dependency; MES, pH 6, r.t., 2.5 h, turnover determined by ratio of MALDI-MS peak intensity ([m/z 2369/(m/z 2207+m/z 2369)]*100) from crude reaction mixture.

FIG. 4 is a column chart showing the galactosylation of a GnGnF⁶ acceptor substrate (dabsyl-GEN[ GnGnF⁶]R)—functionality of the tagged and non-tagged construct; MES, pH 6, r.t., 2.5 h, turnover determined by ratio of MALDI-MS peak intensity ([m/z 2369/(m/z 2207+m/z 2369)]*100) from crude reaction mixture.

FIG. 5 shows the galactosylation of a GnGnF⁶ acceptor substrate (dabsyl-GEN[ GnGnF⁶]R)—functionality of the tagged and non-tagged construct (MES pH 6, r.t., 2.5 h) by way of MS analysis. Upper spectrum: reaction without UDP-Gal, central spectrum: with UDP-Gal, bottom spectrum: digest of the product from the central spectrum with Aspergillus β-galactosidase (citrate buffer, pH 5, r.t., 2 d). The enzyme clearly adds a galactose to this acceptor substrate which can be digested with β-galactosidase, and therefore shows a β-linked Gal residue incorporated by the GalT. Additional GlcNAc removal takes place after prolonged reaction times (>2 d) due to presence of hexosaminidase in the insect cell crude extract.

FIG. 6 is a comparison of MS/MS spectra of acceptor (upper spectrum) and galactosylated reaction product (lower spectrum) of FIG. 5. The MS/MS analysis clearly shows the galactose being linked to the core fucose, as observed from secondary ion 1272.61 corresponding to a Hex-dHex-HexNAc motif linked to the dabsylated GENR peptide.

FIG. 7 is a comparative analysis of the donor specificity of the galactosyl transferase (dansyl-N[GnGnF⁶]ST, MES pH 6.5, Mn²⁺, r.t., 13 h). The enzyme seems to have a high specificity for UDP-Gal, with a negligible residual activity on UDP-Glc.

FIG. 8 is column chart of an analysis of the acceptor specificity: Caenorhabditis elegans GalT galactosylates selectively α-1,6 linked over α-1,3-linked fucose; dabsylGEN-[MMF^{6/3}]R, MES pH 6.5, r.t., 2.5 h, turnover determined by ratio of MALDI-MS peak intensity ([m/z 1963/(m/z 1801+m/z 1963)]*100) from crude reaction mixture.

FIG. 9a shows the graphic determination of the Kₘ (app) of the untagged galactosyl transferase for UDP-Gal: Kₘ (app, UDP-Gal)=ca. 40 μM.

FIG. 9b shows the graphic determination of the Kₘ (app) of the untagged galactosyl transferase for UDP-Gal: Kₘ (app, UDP-Gal)=ca. 40 μM.

FIG. 10 is an analysis of the temperature dependency of the galactosyltransferase of the invention (dansyl-N[GnGnF⁶]ST, UDP-Gal, MES pH 6.5, Mn²⁺, 2.5 h).

FIG. 11 is a column chart demonstrating the glycosylation of human IgG1 (possessing GnGnF⁶ epitopes) with the polypeptide of the invention, i.e. Caenorhabditis elegans core galactosyltransferase.

FIG. 12 is a MALDI-TOF MS spectrum demonstrating the glycosylation of remodelled human transferrin (possessing GnGnF⁶ epitopes) with a polypeptide of the invention, i.e. Caenorhabditis elegans core galactosyltransferase. The indicated m/z values correspond to peptide 622-642 carrying GnGn (3813), GnGnF⁶ (3957) and GnGnF⁶Gal (4119), respectively.

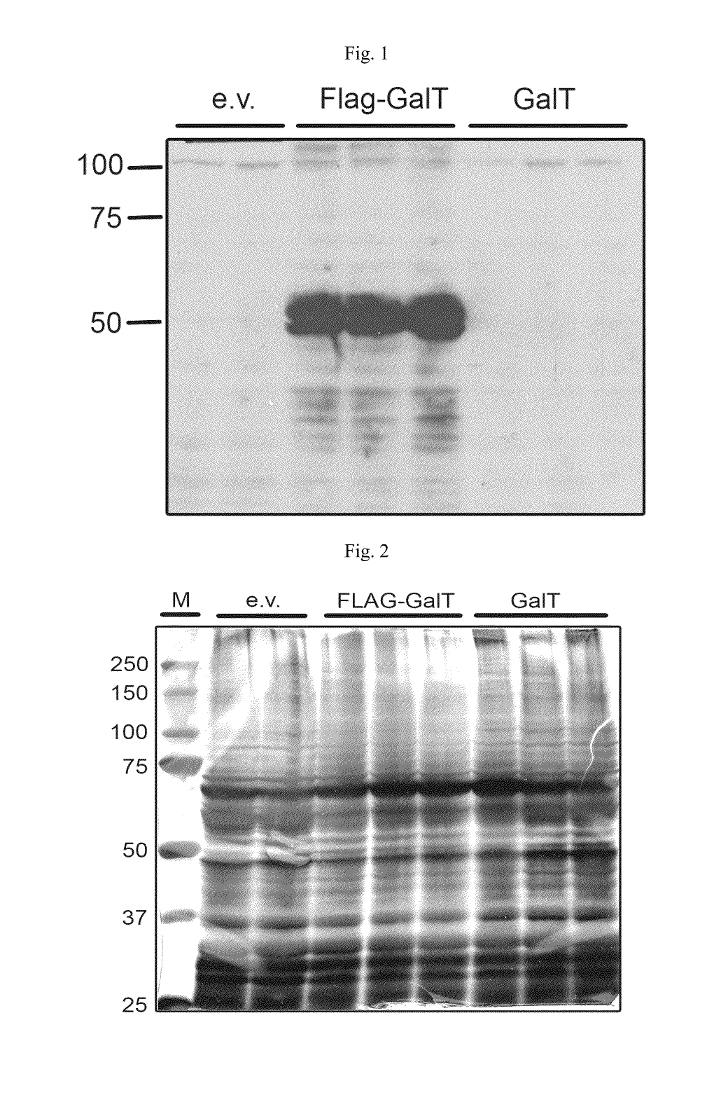

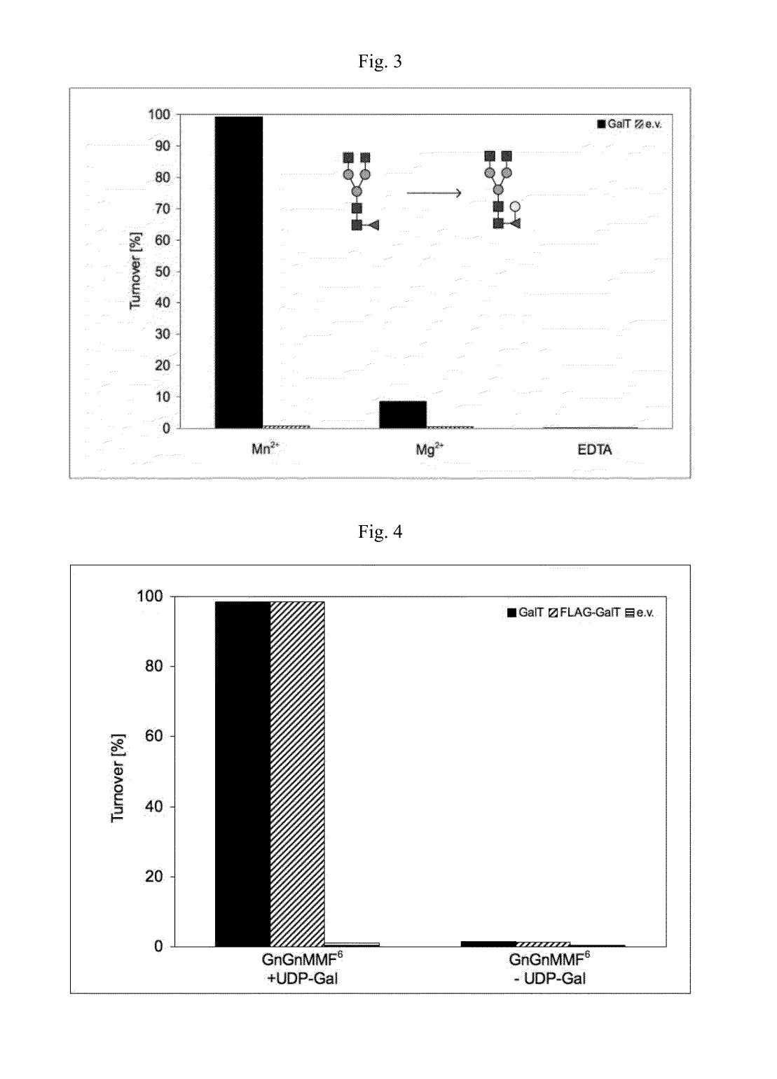

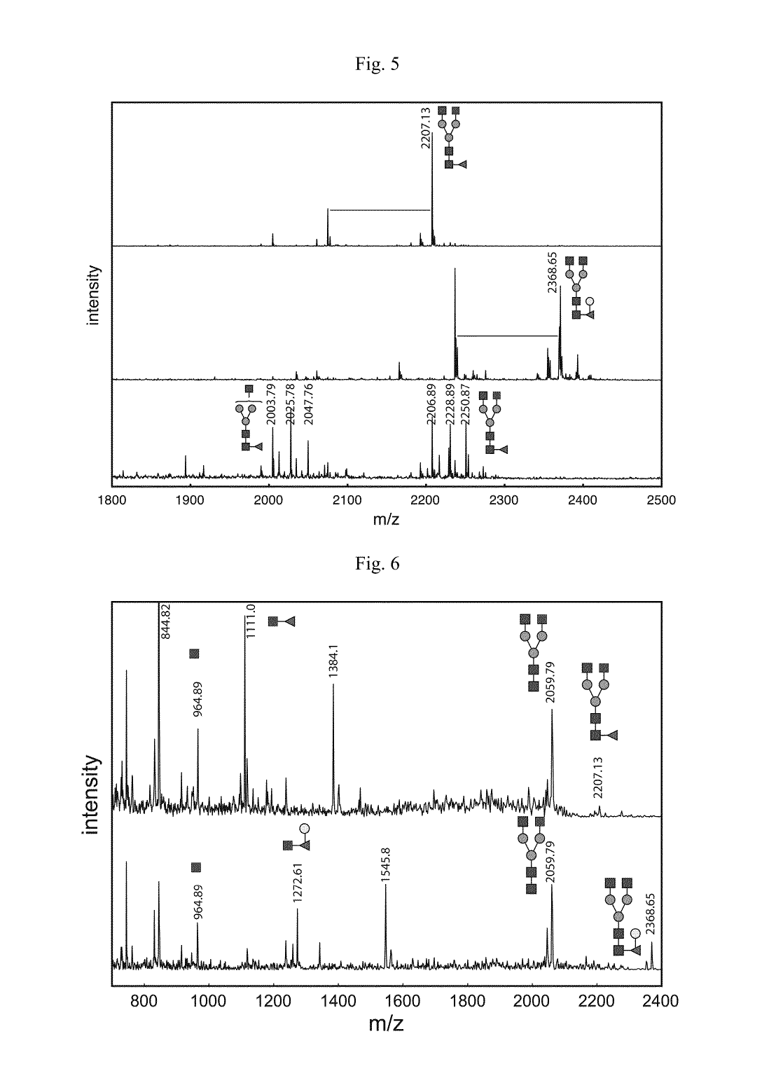

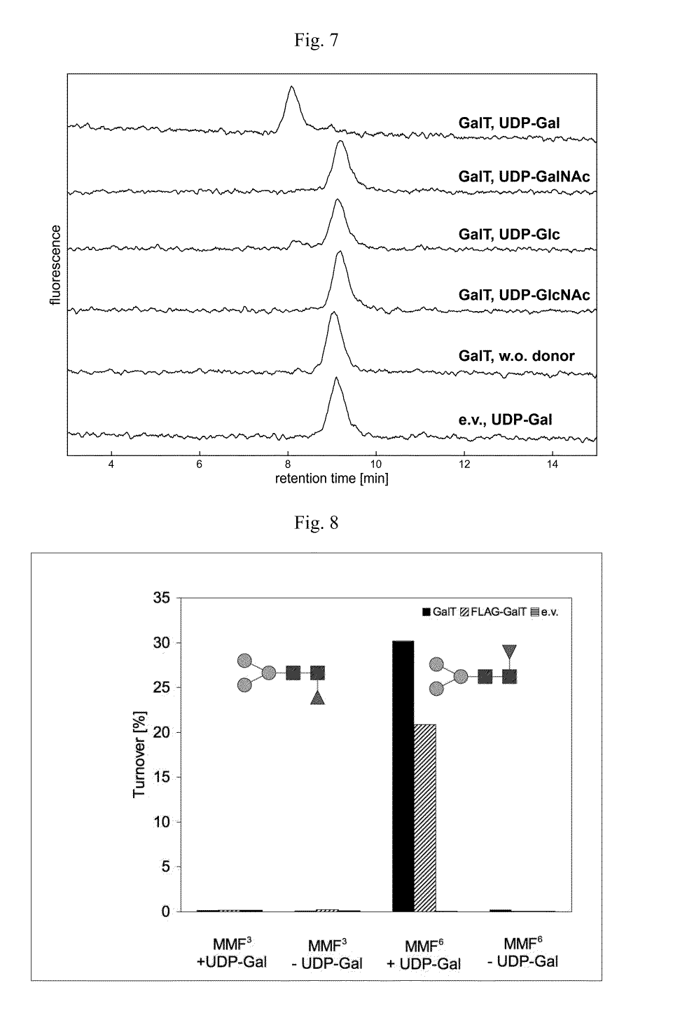

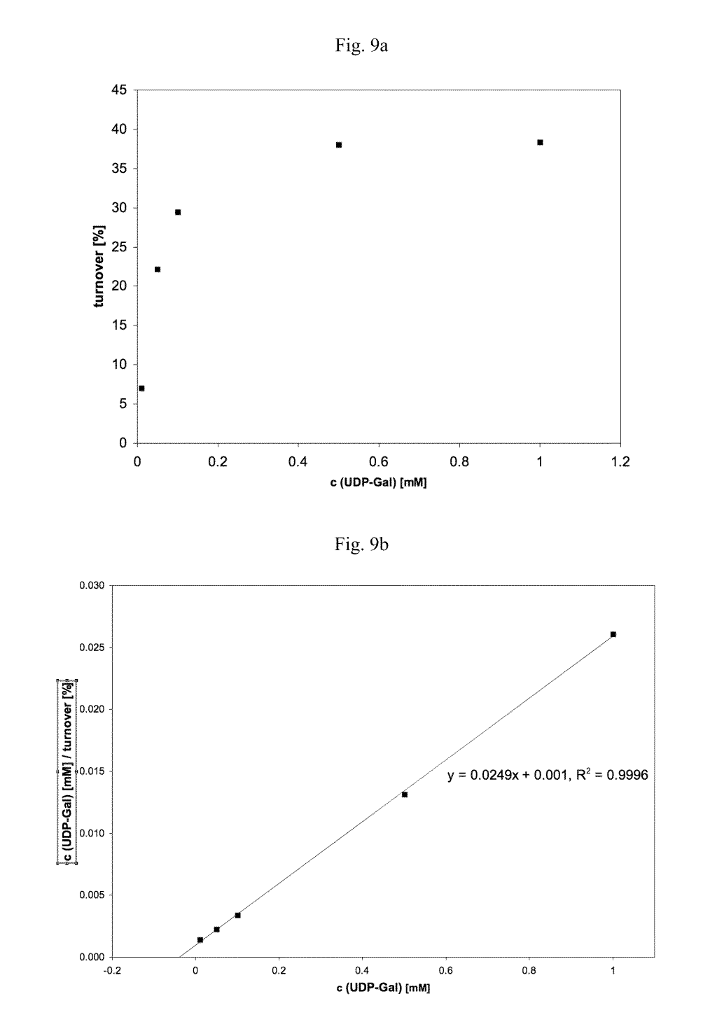

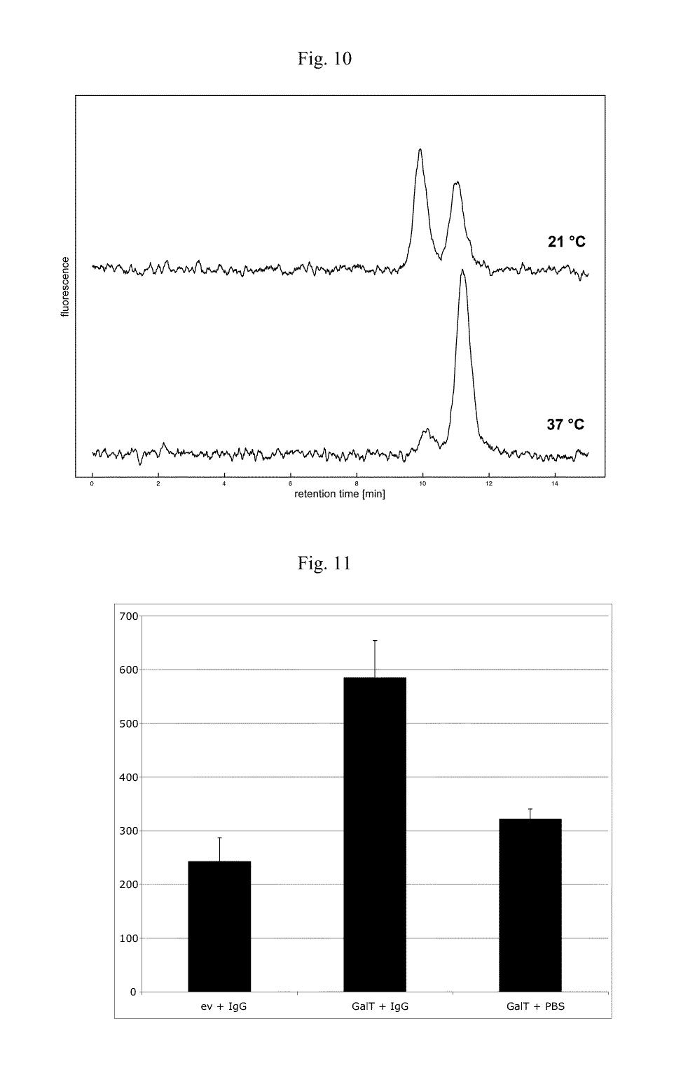

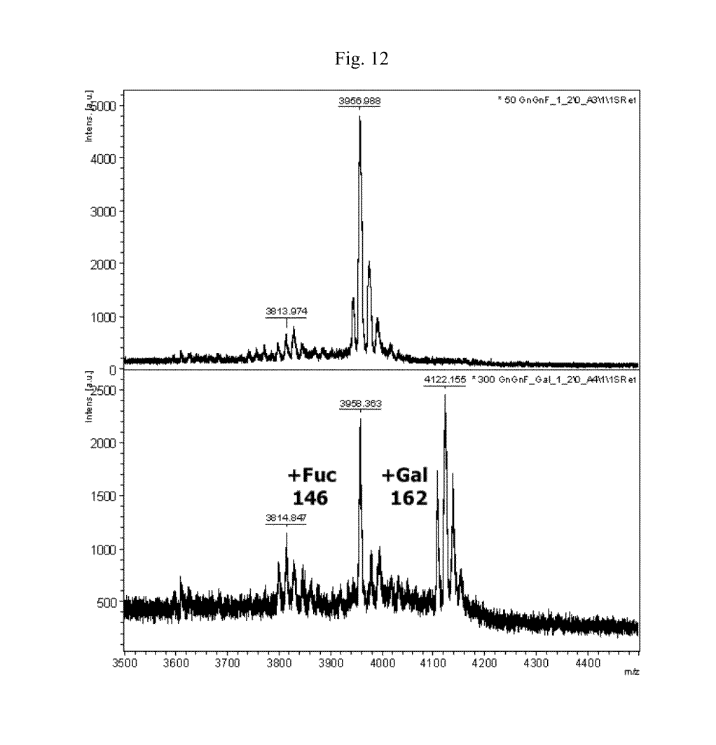

### EXAMPLES

Experimental Procedures

Chemicals and Suppliers

UDP-Gal (VWR International and Sigma), UDP-Glc, UDP-GlcNAc, UDP-GalNAc (all SIGMA), UDP-¹⁴C-Gal (GE Healthcare), GlcNAc-β-1,2-Man-α-1,6-[GlcNAc-β-1,2-Man-α-1,3-1-Man, Man-β-1,4-GlcNAc-β-1,4-[α-1,6-Fuc]-GlcNAc, MMF6, GnGnF⁶ (all Dextra Laboratories, UK), Fuc-α-1,6-GlcNAc (Carbosynth Ltd., UK), dabsyl-GEN[GnGnF⁶]R (Paschinger et al., Glycobiology, 15(5), 463-474, 2005), dabsyl-GEN[MMF6]R (Fabini et al., J. Biol. Chem. 276(30), 28058-28067, 2001), dabsyl-GEN[MMF3]R (Fabini et al., J. Biol. Chem. 276(30), 28058-28067, 2001) and dansyl-N[GnGnF⁶]ST (Roitinger et al., Glycoconj. J., 15(1), 89-91, 1998) were obtained according to previously published methods.

### Example 1

### Isolation of Caenorhabditis elegans cDNA and Cloning of M03F8.4 into Expression Vectors

Nematode Strains:

Methods for culturing Caenorhabditis elegans are described in Brenner, S. (Genetics 77(1), 71-94, 1974). The wild type Bristol N2 strain was grown at 20 ° C. on standard NGM agar plates seeded with Escherichia coli OP50.

Isolation of Caenorhabditis elegans M03F8.4 cDNA:

A Caenorhabditis elegans mixed culture was harvested from one standard NGM agar plate and washed twice in sterile M9 buffer (22 mM KH₂PO₄, 42 mM Na₂HPO₄, 85 mM NaCl, 1 mM MgSO₄). Total RNA was extracted using the NucleoSpin® RNA II RNA isolation kit (MACHEREY-NAGEL AG). cDNA synthesis was performed with 0.5 μg total RNA using the First-strand cDNA synthesis step of the SuperScript™ III Platinum Two-Step qRT-PCR Kit (Invitrogen AG).

Construction of the pFastBac1 Donor Plasmid for Recombinant Gene Expression in sf9 Insect Cells:

M03F8.4 cDNA was isolated from a previously prepared cDNA library by PCR using Phusion High-Fidelity DNA Polymerase (Finnzymes) according to the manual supplied. For construction of an untagged version, the following forward and reverse primers, flanked with SalI and Xbal restrictions sites, respectively, were used: 5′-TTTGTCGA-CACTTCTGAATGCCTCG-3′ (SEQ ID NO: 11) and TTTTCTAGACTACAAGTCTAA-AAGACCAAC-3′ (SEQ ID NO: 12). The resulting fragment was digested with the appropriate restriction enzymes and cloned into the pFastBac1 donor plasmid (Invitrogen). For construction of an N-terminally FLAG tagged version, a forward primer lacking the start codon was used: 5′-TTTGTCGACCCTCGAATCACCGCC-3′ (SEQ ID NO: 13). The resulting fragment was cloned into a pFastBac1 donor plasmid containing an N-terminal FLAG sequence (Muller et al., J. Biol. Chem. 277(36), 32417-32420, 2002) (both vectors kindly provided by Thierry Hennet, Institute of Physiology, University of Zurich).

### Example 2

### Expression of Recombinant Proteins

Recombinant baculoviruses containing the Caenorhabditis elegans core beta-1,4-GalT candidate cDNA (with and without N-terminal FLAG-tag) and an empty vector control were generated according to the manufacturers instructions (Invitrogen). After infection of 2×10⁶ S. frugiperda (sf9) adherent insect cells with recombinant baculoviruses and incubation for 72 h at 28° C., the cells were lysed with shaking (4° C., 15 min) in 150 μL tris-buffered saline (pH 7.4) containing 2% (v/v) Triton-X100 and protease inhibitor cocktail (Roche, complete EDTA-free). The lysis mixtures were centrifuged (2000×g, 5 min) and the postnuclear supernatant was recovered and used for all further enzymatic studies.

### Example 3

### Denaturing Gel Electrophoretic Analysis and Immunoblotting

Infected sf9 cells (2×10⁶ cells, see above) were vortexed in 200 μL Laemmli buffer and proteins denatured by heating (95° C., 5 min). After cooling to r.t. the samples were centrifuged (16 krpm, 5 min) and the supernatant was used for further analysis. The samples were separated by SDS-PAGE (12% acrylamide, 120 V). The resulting gels were either analyzed by silver-staining or by blotting onto a nitrocellulose membrane. After blocking the membrane (5% BSA in PBST) immuno-detection was performed by incubation with anti-FLAG antibody M2 (SIGMA, dilution 1:2000 in PBST+1% BSA) followed by anti-mouse-HRP (Santa Cruz Biotechnology, dilution 1:10000 in PBST+1% BSA) after extensive washing (PBST) and final detection using ECL (Pierce) and exposure to photographic film.

### Example 4

### Glycosyltransferase Assays

Enzymatic activity towards appropriate carbohydrates or glycoconjugates was assessed using 0.5 μL of raw extract of sf9 cells (containing either an empty vector control bacmid, a putative GalT expressing bacmid or a putative FLAG-tagged GalT expressing bacmid) in 2.5 pL final volume of MES buffer (pH 6.5, 40 μM) containing manganese(II) chloride (10 μM), UDP-galactose (1 mM) and the acceptor fucoside (glycan or glyco(poly)peptide, 40 μM). Glycosylation reactions were typically run for 2 h at room temperature, unless noted otherwise. For donor specificity analysis UDP-galactose was replaced by equal concentrations of UDP-Glc, UDP-GlcNAc or UDP-GalNAc (Sigma) respectively. For co-factor-specificity analysis MnCl₂ was replaced by equal concentrations of the various metal chlorides or Na₂EDTA. To quantify the incorporation of galactose into the acceptor glycans total UDP-Gal concentration was doped with 10% UDP-¹⁴C-Gal (25 nCi, GE Healthcare). Excess radioactivity (UDP-¹⁴C-Gal) was removed by loading the reaction mixture (quenched with 100 μL H₂O) onto a column of anion exchange resin (AG1-X8, Cl⁻ form, Bio-Rad Laboratories, 200 mg) and elution of the uncharged products (H₂O, 900 μL).

Glycosylation of human IgG1 (5 μL of 3 g/L, Calbiochem) was performed in 50 μL total volume using the same buffer, salt and enzyme conditions as described above, except the absence of non-radioactive UDP-Gal, which was replaced by UDP-¹⁴C-Gal (75 nCi). The reaction was performed at r.t. over night. A suspension of sepharose-protein G beads (Amersham Biosciences, 10 μL) in PBS (200 μL) was added and binding of IgG1 to the beads was done with shaking (4° C., 1 h). The beads were washed with PBS (5×200 μL) and IgG1 was eluted with 20 mM aqueous HCl (3×100 μL). Analysis (vide infra) of the reaction products was performed either by direct MALDI-TOF mass spectrometry, HPLC analysis of fluorescently labelled glycopeptides for donor specificity or scintillation counting of radio-labelled assays.

Stepwise remodelling of human asialotransferrin N-glycans was performed as follows:

Asialotransferrin (GalGal) was previously prepared by sialidase treatment of human apo-transferrin (lskratsch et al, Anal. Biochem., 368, 133-146, 2009).

To produce asialoagalactotransferrin (GnGn), β1,4-galactosidase (3U, from Aspergillus oryzae) was added to about 1 mg of GalGal and the sample was incubated for 48 hours at 37° C. (total volume 50 μl).

To obtain GnGnF⁶, the sample was brought to a neutral pH with 0.5 μl 1M NaOH, before 50 nmol of GDP-fucose and 15 μl of a preparation of recombinant Anopheles core α1,6-FucT, expressed in Pichia pastoris, were added. The preparation was incubated overnight before another 50 nmol of GDP-fucose and a further 15 μl enzyme (FucT) were added and again incubated overnight at 37° C. In total, approximately 1 mg of GnGnF⁶ was obtained.

To prepare GalFuc-transferrin, 1 μl of a preparation of recombinant Caenorhabditis elegans GalT, 0.2 mmol of MnCl₂ and 20 nmol of UDP-galctose were added to an aliquot of GnGnF⁶ (300 μg) and incubated overnight at 30° C. Again, the desired glycan structure was boosted with a second incubation overnight after the addition of further substrate (UDP-galactose) and enzyme (GalT).

The degree of modification of the transferrin was monitored by dot blotting with the fucose-specific Aleuria aurantia lectin and by MALDI-TOF MS of tryptic peptides of the various neoglycoforms.

### Example 5

### Structural Analysis

After exposing dabsyl-GEN[GnGnF⁶]R to galactosylation conditions, the resulting crude mixture was adjusted to 50 mM sodium citrate and pH 4.5, digested with Aspergillus oryzae β-galactosidase (27 mU) (see Gutternigg et al., J. Biol. Chem. 282(38), 27825-27840, 2007) for 2 days at 30° C. The samples were analyzed by MALDI-TOF mass spectrometry (vide infra).

HPLC Analysis:

Both, for analysis of donor specificity and the reaction rate dependence on donor concentration, the dansyl-N[GnGnF⁶]ST acceptor substrate was separated from the galactosylated reaction product using an isocratic solvent system (0.7 mL/min, 9% MeCN (95%, (v/v)) in 0.05% aqueous TFA (v/v)) on a reversed phase Hypersil ODS C18 column (4×250 mm, 5 μm) and fluorescence detection (excitation at 315 nm, emission detected at 550 nm) at room temperature. The Shimadzu HPLC system consisted of a SCL-10A controller, two LC10AP pumps and a RF-10AXL fluorescence detector controlled by a personal computer using Class-VP software (V6.13SP2). Dansyl-N[ GnGnF⁶]ST eluted at a retention time of 9.09 min and the galactosylated reaction product at 8.06 min.

Mass Spectrometry:

Glycans were analyzed by MALDI-TOF mass spectrometry on a BRUKER Ultraflex TOF/TOF machine using a α-cyano-4-hydroxy cinnamic acid matrix. A peptide standard mixture (Bruker) was used for external calibration.

Scintillation Counting:

The eluates of the anion exchange resin column and protein G beads were thoroughly mixed with scintillation fluid (Irga-Safe Plus, Packard, 4 mL) and measured with a Perkin Elmer Tri-Carb 2800TR.

Abbreviations for Carbohydrates:

Fuc—L-fucose, Gal—D-galactose, GalNAc—D-N-acetylgalactosamine, Glc—D-glucose, GlcNAc—D-N-acetylglucosamine, Man—D-mannose

Abbreviations for complex glycans (according to the Schachter nomenclature [Biochem Cell Biol 64(3), 163-181, 1986]):
GalGal Gal-β-1,4-GlcNAc-β-1,2-Man-α-1,6-[Gal-β-1,4-GlcNAc-β-1,2-Man-α-1,3-]-Man-β-1,4-GlcNAc-β-1,4-GlcNAc
GnGn GlcNAc-β-1,2-Man-α-1,6-[GlcNAc-β-1,2-Man-α-1,3-]-Man-β-1,4-GlcNAc-β-1,4-GlcNAc
GnGnF⁶ GlcNAc-β-1,2-Man-α-1,6-[GlcNAc-β-1,2-Man-α-1,3-]-Man-β-1,4-GlcNAc-β-1,4-[α-1,6-Fuc]-GlcNAc
GnGnF⁶Gal GlcNAc-β-1,2-Man-α-1,6-[GlcNAc-β-1,2-Man-α-1,3-]-Man-β-1,4-GlcNAc-β-1,4-[Gal-β-1,4-Fuc-α-1,6]-GlcNAc
MMF⁶ Man-α-1,6-[Man-α-1,3-]-Man-β-1,4-GlcNAc-β-1,4-[α-1,6-Fuc]-GlcNAc
MMF⁶Gal Man-α-1,6-[Man-α-1,3-]-Man-β-1,4-GlcNAc-β-1,4-[Gal-β-1,4-Fuc-α-1,6]-GlcNAc
MMF³ Man-α-1,6-[Man-α-1,3-]-Man-β-1,4-GlcNAc-β-1,4-[α-1,3-Fuc]-GlcNAc

1-15. (canceled)

16. An isolated and purified polypeptide selected from the group consisting of:
(a) polypeptides having an amino acid sequence selected from the group consisting of SEQ ID NOs: 2, 4, 6, 8 and 10,
(b) polypeptides encoded by a nucleic acid having a sequence selected from the sequences listed in SEQ ID NOs: 1, 3, 5, 7 and 9,
(c) polypeptides encoded by a nucleic acid having a sequence with at least 90% identity to the sequence listed in SEQ ID NO 1,
(d) polypeptides encoded by a nucleic acid that hybridizes to a nucleic acid having a sequence selected from the sequences listed in SEQ ID NOs: 1, 3, 5, 7 and 9 under stringent conditions of 6× sodium chloride/sodium citrate (SSC) at 45° C. followed by a wash in 0.2×SSC, 0.1 SDS at 65° C., and
(e) polypeptides having an amino acid sequence identity of at least 90 or 95% with the polypeptides of (a) to (d),
wherein said polypeptide has galactosyltransferase activity.

17. The polypeptide of claim 16, wherein said polypeptide has β-1,4-galactosyltransferase activity.

18. The polypeptide of claim 16, wherein said polypeptide has β-1,4-galactosyltransferase activity with L-fucoside-, α-L-fucoside- or Fuc-α-1,6-GlcNAc, or GnGnF⁶-containing poly/oligosaccharides or glycoconjugates as acceptor substrates.

19. An isolated and purified polypeptide having an amino acid sequence selected from the group consisting of SEQ ID NOs 2, 4, 6, 8, and 10.
